(19) United States
(12) Patent Application Publication
(10) Pub. No.: US 2005/0020542 A1
(43) Pub. Date: Jan. 27, 2005

(54) Method of modulating sodium ion absorption in epithelial cells

(76) Inventors: Alexis Traynor-Kaplan, North Bend, WA (US); Mark Moody, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/490,222

(22) PCT Filed: Sep. 19, 2002
(86) PCT No.: PCT/US02/29965

Related U.S. Application Data
(60) Provisional application No. 60/323,953, filed on Sep. 20, 2001.

(51) Int. Cl. A61K 31/66

(52) U.S. Cl. 514/102

(57) ABSTRACT

The invention provides methods for modulating sodium ion absorption by epithelial cells, by treating epithelial cells or administering to a patient in need of such treatment a therapeutically effective amount of a sodium uptake modulating inositol polyphosphate compound. The sodium uptake modulating inositol polyphosphate compound can be designed to inhibit or enhance sodium uptake. Representative sodium uptake inhibiting inositol polyphosphate compounds include, for example, 1-octyl-2-O-bu-tyryl-inositol 3,4,5,6-tetrakisphosphate propionoxymethyl ester (INO E2).

### FIELD OF THE INVENTION

The present invention relates to inositol derivatives that modulate the absorption of sodium ions in epithelial cells, such as in mucosal epithelia of patients suffering from cystic fibrosis. The present invention also relates to methods for regulating the epithelial sodium channel (ENaC) using effective inositol polyphosphate compounds, alone or in combination with other therapeutic agents, such as for treating pathological conditions related to cystic fibrosis, regulating fluid retention and/or regulating blood pressure in humans.

### BACKGROUND OF THE INVENTION

Cystic fibrosis (CF) is the most common genetic disorder and the largest genetic killer of children. One in twenty Caucasians carries a defective CF gene, which, when coupled with a spouse who is also a carrier can result in offspring afflicted with CF. An autosomal, recessive disorder, one in 3,000 children born in the United States and Europe inherit CF. Children live for varying periods of time, but the average has been extended from a couple of years early in this century to a current life expectancy of 30 years. Over 70,000 patients have been identified with Cystic Fibrosis worldwide. This translates into over 30,000 individuals with the disease in the United States with another 30,000 who have been identified with the disorder in Europe. As current treatment strategies prolong the average lifespan, the number of CF patients is expected to rise. Patients with CF typically incur medical costs ranging from $15,000 to $55,000 annually.

The disease causes abnormally viscous mucous secretions that lead to chronic pulmonary disease, pancreatic insufficiency and intestinal obstructions, together with a host of lesser but potentially lethal problems, such as an excessive loss of electrolytes in hot environments. In the past, afflicted children often died as infants. Although surviving into their twenties and thirties with current treatments, CF patients are plagued with recurrent infections and require daily arduous routines to clear air passageways.

In CF, mutations in the gene coding for the Cystic Fibrosis Transmembrane Conductance Regulator (CFTR) protein result in defective Cl⁻ transport. The defect in the CFTR is also linked to hyperabsorption of Na⁺ through the epithelial sodium channel (ENaC) (Boucher et al., 1986; Greger, 2000; Knowles et al., 1986; Mall et al., 1999) which is believed to account for an elevated basal short-circuit current (I_{sc}) in CF mucosal epithelia and further to exacerbate the defect. This combination of ion transport abnormalities results in a reduced capacity to control airway surface liquid volume and reduced mucocilliary clearance, contributing to the pathophysiological conditions presenting in CF airways (Matsui et al., 2000; Matsui et al., 1998). The effort to correct the defective ion transport associated with CF has focused on the mechanisms modulating ENaC, CFTR, and alternate Cl⁻ channel function. There are compelling arguments for pursuing artificial activation of alternate Cl⁻ channels to counteract CF pathophysiology. Mucosal epithelia express C⁻ channels other than the CFTR such as the outwardly rectifying chloride channel (ORCC), calcium activated Cl⁻ channels (CLCA) and volume regulated Cl⁻ channels. All are potential targets for CF treatment. In fact, the ORCC may also be controlled by the CFTR and therefore be dysfunctional in CF (Clarke et al., 1994; Egan et al., 1992; Gabriel et al., 1993; Schwiebert et al., 1995). In contrast, Ca²⁺-dependent Cl⁻ channels are reportedly more abundant in CF tissue (Grubb et al., 1994). A number of studies indicate that phenotypes with increased activity of alternate Cl⁻ channels such as the Ca²⁺ dependent Cl⁻ channels correlate with milder clinical manifestations, (Clarke et al., 1994; Leung et al., 1995; Pilewski and Frizzell, 1999; Rozmahel et al., 1996; Veeze et al., 1994). Stimulation of apical Cl⁻ secretion through the CFTR and Ca²⁺ activated Cl⁻ channels has recently been found to be closely associated with ENaC function and sodium absorption in mucosal epithelia (Devor and Pilewski, 1999; Inglis et al., 1999; Mall et al., 1999; Ramminger et al., 1999; Wang and Chan, 2000). Thus, it has been hypothesized that alternate Cl⁻ channels such as the Ca²⁺ activated Cl⁻ channel and the ClC-x family may compensate for defects in CFTR function and could be utilized in a therapeutic strategy. This has lead to efforts to probe the usefulness of agents that elevate intracellular Ca²⁺, such as purinergic agonists, in the treatment of CF (Bennett et al., 1996). Currently two compounds are in development because they elevate intracellular calcium and thereby modulate Cl⁻ secretion, INS365—a PY2Y receptor agonist, and duramycin—an antibiotic that triggers an increase in intracellular calcium levels.

However, an increase in intracellular Ca²⁺ does not always lead to Cl⁻ secretion. It has been demonstrated that the intracellular signaling molecule, inositol 3,4,5,6 tetrakisphosphate (Ins(3,4,5,6)P₄) “uncouples” chloride secretion from the rise in intracellular calcium in mucosal epithelia (Vajanaphanich, et al. 1994). This regulatory role for Ins(3,4,5,6)P₄ has been confirmed by several investigators (Ho et al., 1997; Xie et al., 1998, Ismailov, et al., 1996).

Despite the foregoing advances, a need exists for new and improved methods for regulating ion transport in epithelial cells, such as by the modulation of ENaC.

### SUMMARY OF THE INVENTION

It has now been discovered that sodium ion absorption by epithelial cells can be modulated by administering to a patient in need of such treatment a therapeutically effective amount of a sodium uptake modulating inositol polyphosphate compound, or a racemate thereof, or a pharmaceutically acceptable salt thereof. In one aspect, the invention provides methods for inhibiting sodium ion absorption by epithelial cells, comprising administering to a patient in need of such treatment a therapeutically effective amount of a sodium uptake inhibiting inositol polyphosphate compound, such as 1-octyl-2-O-butyryl-inositol 3,4,5,6-tetrakisphosphate propionoxymethyl ester (INO E2), or a racemate or a pharmaceutically acceptable salt thereof. In another aspect of the invention, the invention provides methods for enhancing sodium ion absorption by epithelial cells, comprising administering to a patient in need of such treatment a therapeutically effective amount of a sodium uptake enhancing inositol polyphosphate compound, such as 1,4-di-O-butyryl-inositol 2,3,5,6-tetrakisphosphate propionoxymethyl ester, or a racemate or a pharmaceutically acceptable salt thereof.

Because Ins(3,4,5,6)P₄ inhibits Ca²⁺-dependent Cl⁻ secretion in colonic epithelia and a CF pancreatic epithelial cell line, CFPAC-1 (Carew and Thorn, 2000; Carew et al., 2000; Vajanaphanich et al., 1994), it has been discovered that certain analogues of Ins(3,4,5,6)P₄ are stimulatory. Such molecules appear to act “downstream” of the rise in intracellular Ca²⁺.

Ins(3,4,5,6)P₄ analogues have been constructed in order to identify molecules that activate alternate Cl⁻ channels. These analogues and their effects on Cl⁻ transport were investigated in Ussing chambers using monolayer cultures of primary CF Human nasal epithelial cells that have been shown, in early passages, to reflect in vivo characteristics. Additionally, currents activated by certain Ins(3,4,5,6)P₄ analogues in individual primary CF human nasal epithelial cells have been identified by whole cell patch, clamp technique.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a graph showing the acute effect of 2-O-butyryl-1-O-octyl-myo-inositol 3,4,5,6-tetrakisphosphate octakis (propionoxymethyl) ester (INO-E2) on basal short-circuit current (I_{sc}) and resistance in cystic fibrosis human nasal epithelial (CFHNE) cells, as described in Example 1. FIGS. 1A and 1B depict the effect of acute addition of INO-E2 on I_{sc} and resistance in CFHNE monolayers. FIG. 1C depicts the dose response of the inhibitory effect of INO-E2 on I_{sc} expressed as time to 50% inhibition.

FIG. 2 is a graph showing the dose-dependence of the long-term effect (2 hour incubation, 24 hours prior to recording) of INO-E2 on spontaneous I_{sc} and resistance in CFHNE. FIG. 2A shows the difference in basal amiloride-inhibitable I_{sc} in monolayers incubated with two different concentrations of INO-E2 vs. vehicle control. FIG. 2B depicts the dose-dependence of the INO-E2 inhibition of basal amiloride-inhibitable I_{sc}. FIG. 2C depicts the corresponding dose dependence of the effect of INO-E2 on monolayer resistance.

FIG. 3 is a graph showing the effect of repeated exposure to INO-E2 on physiological parameters in CFHNE. 2.5 μM INO-E2 was added every 24 hrs for 4 days. Effects on I_{sc} in CFHNE, passage 2 are depicted. Every 24 hrs medium in the basolateral compartment was exchanged with fresh medium containing 2.5 μM INO-E2. On the 4th day the monolayers were mounted in Ussing chambers and basal I_{sc}, conductance and resistance measured. After a stable baseline was reached, amiloride was added to determine the amiloride-inhibitable-I_{sc}. Under these conditions, subsequent apical addition of the Ca²⁺-mobilizing agent, ATP, allows Cl⁻ secretion to be examined in isolation. FIG. 3A shows I_{sc} in μA/cm²; FIG. 3B shows resistance (R) in ohms cm²; FIG. 3C shows basal P.D. in mV; and FIG. 3D shows the % increase in Cl⁻ secretion measured over 15 min.

FIG. 4 is a graph showing the effect of 24-hour pre-incubation (2 hr exposure) and acute addition with 5 μM D-PIP₃/AM on I_{sc} in cultured CF human nasal epithelial cells, using the procedure generally described in Example 1. Note the effect of preincubation in the first 25 minutes of the recording.

FIG. 5 is a graph showing the effect of 24-hour pre-incubation (2 hr exposure) and acute addition with 5 μM Bt₂-inositol 2,3,4,5-tetrakisphosphate propionoxymethyl ester on I_{sc} in cultured CF human nasal epithelial cells, using the procedure generally described in Example 1.

FIGS. 6A and 6B are graphs showing (6A) the effect of 24 hr. pre-incubation (2 hr. exposure) with 1 μM 2,6-Di-O-butyryl-myo-inositol 1,3,4,5-tetrakisphosphate octakis (propionoxymethyl) (INO-230) ester on I_{sc} in cultured CF human nasal epithelia, passage 3 (5CFHNEP3), and (6B) the effect of 24 hr. pre-incubation (2 hr. exposure) with 1 μM and 20 μM 2-O-butyryl-1-O-octyl-myo-inositol 3,4,5,6-tetrakisphosphate octakis (propionoxymethyl) ester (INO-E2).

FIG. 7A is a graph showing the effect of 24 hr. pre-incubation (2 hr. exposure) with 10 μM 1,2,6 tri-O-butyl-myo-inositol 3,4,5 trisphosphate hexakis (propionoxymethyl)ester on I_{sc} in cultured CF human nasal epithelia, passage 3 (4CFHNEP3).

FIG. 7B is a graph showing the effect of 24 hr. pre-incubation (2 hr. exposure) with 200 μM PI(3,4)P₂/AM on I_{sc} in cultured CF human nasal epithelia, passage 3 (4CFHNEP3).

FIG. 8 is a graph showing the dose dependent inhibition of fluid absorption by 1,2,5-tri-O-butyryl-myo-inositol 3,4,6-trisphosphate hexakis (propionoxymethyl) ester (ent-TMX/PM; INO-4981) using the Blue Dextran Assay. Amil.: 100 micromolar amiloride.

FIG. 9 is a graph showing the dose dependent inhibition of fluid absorption by 2,3,5-tri-O-butyryl-myo-inositol 1,4,6-trisphosphate hexakis (propionoxymethyl) ester (TMX/PM; INO-4982) using the Blue Dextran Assay. Amil.: 100 micromolar amiloride.

FIG. 10 is a graph showing the dose dependent inhibition of fluid absorption by 2,3-camphanylidene-myo-inositol 1,4,5,6-tetrakisphosphate octakis (propionoxymethyl) ester (INO-4984) using the Blue Dextran Assay. Amil.: 100 micromolar amiloride.

FIG. 11 is a graph showing the dose dependent inhibition of fluid absorption by 2,4,6-tri-O-butyryl-myo-inositol 1,3,5-trisphosphate hexakis (propionoxymethyl) ester (INO-4992) using the Blue Dextran Assay. Amil.: 100 micromolar amiloride.

FIG. 12 is a graph showing the dose dependent inhibition of fluid absorption by 2-O-butyryl-3-O-octyl-myo-inositol 1,4,5,6-tetrakisphosphate octakis (propionoxymethyl) ester (INO-E3; INO-4987) using the Blue Dextran Assay. Amil.: 100 micromolar amiloride.

FIG. 13 is a graph showing the dose dependent inhibition of fluid absorption by 2-O-butyryl-1-O-octyl-myo-inositol 3,4,5,6-tetrakisphosphate octakis (propionoxymethyl) ester (INO-E2; INO-4995) using the Blue Dextran Assay. Amil.: 100 micromolar amiloride.

The Lower Dotted line represents the effects of 100 μM amiloride.

FIG. 14 is a graph showing the dose dependent inhibition of fluid absorption by 2-O-butyryl-1-O-(3-phenylpropyl)-myo-inositol 3,4,5,6-tetrakis-phosphate octakis (propionoxymethyl) ester (INO-4997) using the Blue Dextran Assay. Amil.: 100 micromolar amiloride.

FIG. 15 is a graph showing the dose dependent inhibition of fluid absorption by 2,6-Di-O-butyryl-myo-inositol 1,3,4,5-tetrakisphosphate octakis(propionoxymethyl) ester (INO-4991) using the Blue Dextran Assay. Amil.: 100 micromolar amiloride.

FIG. 16 is a graph showing the dose dependent inhibition of fluid absorption by 1,2-camphanylidene-myo-inositol 3,4,5,6-tetrakisphosphate octakis (propionoxymethyl) ester (INO-4996) using the Blue Dextran Assay. Amil.: 100 micromolar amiloride.

FIG. 17 is a graph showing enhancement of compound potency on fluid absorption with repeat administration. Monolayers were exposed to INO-E2 or INO-230 for 2 hours each day for 8 days at concentrations of either 0.1 μM or 1 μM. Data are means+/−SEM for n=6. This graph is representative of 2 separate experiments with similar results.

### DETAILED DESCRIPTION OF THE PREFERRED EMBODIMENT

The invention provides methods for modulating sodium ion absorption by cells, comprising administering to a patient in need of such treatment a therapeutically effective amount of a sodium uptake modulating inositol polyphosphate compound, or a racemate thereof, or a pharmaceutically acceptable-salt thereof. In one aspect of the invention, the invention provides methods for inhibiting sodium ion absorption by epithelial cells, comprising administering to a patient in need of such treatment a therapeutically effective amount of a sodium uptake inhibiting inositol polyphosphate compound, or a racemate thereof, or a pharmaceutically acceptable salt thereof. In another aspect of the invention, the invention provides methods for enhancing sodium ion absorption by epithelial cells, comprising administering to a patient in need of such treatment a therapeutically effective amount of a sodium uptake enhancing inositol polyphosphate compound, or a racemate thereof, or a pharmaceutically acceptable salt thereof.

Sodium uptake inhibiting and enhancing inositol polyphosphate compounds are determined by the cystic fibrosis human nasal epithelial (CFHNE) cell assay, as described in detail in Examples 1 and 2, i.e., by mounting monolayers of human CF nasal epithelial cells in Ussing chambers, and then monitoring short-circuit current (I_{sc}) and resistance after contact with a test inositol polyphosphate compound. Sodium uptake inhibiting inositol polyphosphate compounds generally exhibit reduced I_{sc}, and increased resistance relative to controls. Sodium uptake enhancing inositol polyphosphate compounds generally exhibit increased I_{sc}, and decreased resistance relative to controls.

Sodium uptake inhibiting inositol polyphosphate compounds useful in the practice of the invention include any inositol polyphosphate compounds that inhibit I_{sc} and increase resistance relative to controls as determined by the CFHNE cell assay. Presently preferred sodium uptake inhibiting inositol polyphosphate compounds for use in the practice of the invention include, for example, 2-O-butyryl-1-O-octyl-myo-inositol 3,4,5,6-tetrakisphosphate octakis (propionoxymethyl) ester (INO-E2), 2-O-butyryl-3-O-octyl-myo-inositol 1,4,5,6-tetrakisphosphate octakis (propionoxymethyl) ester (INO-E3), 2-O-butyryl-1-O-(3-phenylpropyl)-myo-inositol 3,4,5,6-tetrakisphosphate octakis (propionoxymethyl) ester, 2,4,6-tri-O-butyryl-myo-inositol 1,3,5-trisphosphate hexakis (propionoxymethyl) ester, 1,2,5-tri-O-butyryl-myo-inositol 3,4,6-trisphosphate hexakis (propionoxymethyl) ester (ent-TMX/PM), 2,3,5-tri-O-butyryl-myo-inositol 1,4,6-trisphosphate hexakis (propionoxymethyl) ester (TMX/PM), 2,3-camphanylidene-myo-inositol 1,4,5,6-tetrakisphosphate octakis (propionoxymethyl) ester, 1,2-camphanylidene-myo-inositol 3,4,5,6-tetrakisphosphate octakis (propionoxymethyl) ester, inositol 3,4,5,6-tetrakisphosphate propionoxymethyl ester, inositol 1,4,5,6-tetrakisphosphate propionoxymethyl ester, D,L-2-O-butyryl-phosphatidylinositol 3,4,5-trisphosphate heptakis(acetoxy)methyl ester (BtPIP₃/AM), 3,6-di-O-butyryl-myo inositol 1,2,4,5-tetrakisphosphate octakis (propionoxymethyl) ester, and 1,4-di-O-butyryl-myo inositol 2,3,5,6-tetrakisphosphate octakis (propionoxymethyl) ester. Other alkylated derivatives of the forgoing compounds may also be used, such as the hexyl, dodecanyl, heptyl, butyl, isopropyl, and isobutyl counterparts of 1-octyl INO E2 or of the other compounds described herein. The presently most preferred sodium uptake inhibiting inositol polyphosphate compound is 1-octyl-2-O-butyryl-inositol 3,4,5,6-tetrakisphosphate propionoxymethyl ester (INO E2), whose activity is described in detail herein. Sodium uptake enhancing inositol polyphosphate compounds useful in the practice of the invention include any inositol polyphosphate compounds that increase I_{sc} and decrease resistance relative to controls as determined by the CFHNE cell assay. Presently particularly preferred sodium uptake enhancing inositol polyphosphate compounds, as determined by the CFHNE cell assay include, for example, 1,4-di-O-butyryl-myo-inositol 2,3,5,6-tetrakisphosphate propionoxymethyl ester.

In presently particularly preferred embodiments, the sodium ion modulators of the invention are designed to be delivered intracellularly, such as by concealing the negatively charged phosphate groups with bioactivatable esters, such as acetoxymethylesters (AM-esters), propionoxymethylesters (PM-esters) or pivaloyloxymethyl esters, and the hydroxy groups with alkyl groups, such as butyrates, where necessary. These masking groups have previously been shown to permit passive diffusion of the inositol polyphosphate compounds across the plasma membrane to the interior of the cell where esterases cleave them and liberate the biologically active inositol polyphosphate inside the cell. (See M. Vajanaphanich et al., Nature 371:711 (1994); Rudolf, M. T. et al., “2-Deoxy derivative is a partial agonist of the intracellular messenger inositol 3,4,5,6-tetrakisphosphate in the epithelial cell line T84” J Med Chem 41:3635-44 (1998)).

Compounds of the invention may be tested in vivo to demonstrate efficacy of the compounds in remediating the symptoms of cystic fibrosis and/or cardiovascular disease. For example, indices measured in vivo that demonstrate the efficacy of compounds include measurement of the effects of the compounds in animals such as mice and human beings in nasal potential difference (NPD) as described in Knowles, M. R., Paradiso, A. M., and Boucher, R. C. (1995). In vivo nasal potential difference: techniques and protocols for assessing efficacy of gene transfer in cystic fibrosis. Hum Gene Ther 6, 445-55; mucociliary clearance of [^{99m}Tc] iron oxide particles as described in Bennett, W. D., Olivier, K. N., Zeman, K. L., Hohneker, K W., Boucher, R. C., and Knowles, M. R. (1996). Effect of uridine 5′-triphosphate plus amiloride on mucociliary clearance in adult cystic fibrosis. Am J Respir Crit Care Med 153,1796-801 and Olivier, K N., Bennett, W. D., Hohneker, K. W., Zeman, K. L., Edwards, L. J., Boucher, R. C., and Knowles, M. R. (1996). Acute safety and effects on mucociliary clearance of aerosolized uridine 5′-triphosphate+/−amiloride in normal human adults. Am J Respir Crit Care Med 154, 217-23; forced expiratory volume 1 (FEV1); measurement of the production of inflammatory mediators and cytokines such as leukotrienes, interleukins, complement factors and platelet activating factor as described in Coffer, P. J., Geijsen, N., M'Rabet, L., Schweizer, R. C., Maikoe, T., Raaijmakers, J. A., Lammers, J. W., and Koenderman, L. (1998). Comparison of the roles of mitogen-activated protein kinase and phosphatidylinositol 3-kinase signal transduction in neutrophil effector function. Biochem J 329, 121-30, and Gibbs; B. F., Schmutzler, W., Vollrath, I. B., Brosthardt, P., Braam, U., Wolff, H. H., and Zwadlo-Klarwasser, G. (1999). Ambroxol inhibits the release of histamine, leukotrienes and cytokines from human leukocytes and mast cells. Inflamm Res 48, 86-93. Such tests as well as a complete blood count show whether secondary infections and ensuing inflammatory responses are ameliorated by treatment. Blood pressure can also be monitored. For determining whether extrapulmonary manifestations are corrected, fecal fat can be evaluated.

The compounds of the present invention can be used in the form of salts derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, persulfate, 3-phenylproionate, picrate, pivalate, propionate, succinate, tartrate, thiocyanate, p-toluenesulfonate and undecanoate.

Examples of acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. Basic addition salts can be prepared in situ during the final isolation and purification of the compounds of formulas (I)-(V), or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

The compounds of the invention are useful in vitro for modulating sodium ion absorption in a cell or tissue, and in vivo in human and animal hosts for the regulation of the sodium channel, ENaC. The compounds may be used alone or in compositions together with a pharmaceutically acceptable carrier.

Thus, in one aspect, the present invention provides methods of treatment of cystic fibrosis in a subject in need of such treatment by administering an inositol polyphosphate as given above to the subject in an amount effective to modulate epithelial sodium ion absorption. In another aspect, the present invention provides methods of treating chronic bronchitis in a subject in need of such treatment by administering an inositol polyphosphate as given above to the subject in an amount effective to modulate epithelial sodium ion absorption. In another aspect, the present invention provides methods of treating asthma in a subject in need of such treatment by administering an inositol polyphosphate analog as given above to the subject in an amount effective to modulate epithelial sodium ion absorption. In another aspect, the present invention provides methods of combating chronic obstructive pulmonary disorder by administering an inositol polyphosphate analog as given above to said subject in an amount effective to modulate epithelial sodium ion absorption. In another aspect, the present invention provides methods of regulating fluid-retention by administering an inositol polyphosphate analog as given above to the subject in an amount effective to modulate epithelial sodium ion absorption. In another aspect, the present invention provides methods of regulating blood pressure by administering an inositol polyphosphate analog as given above to said subject in an amount effective to modulate epithelial sodium ion absorption. In yet other aspects, the present invention provides methods of use of an the active compounds as disclosed herein for the manufacture of a medicament for the prophylactic or therapeutic treatment of cystic fibrosis in a subject in-need of such treatment. In yet other aspects, the present invention provides methods of use of the active compounds as disclosed herein for the manufacture of a medicament for the prophylactic or therapeutic treatment of chronic bronchitis in a subject in need of such treatment. In yet other aspects, the present invention provides methods of use of an the active compounds as disclosed herein for the manufacture of a medicament for the prophylactic or therapeutic treatment of asthma in a subject in need of such treatment.

When administered to a patient, e.g., a mammal for veterinary use or to a human for clinical use, the inositol derivatives are preferably administered in isolated form. By “isolated” is meant that prior to formulation in a composition, the inositol derivatives are separated from other components of either (a) a natural source such as a plant or cell culture, or (b) a synthetic organic chemical reaction mixture. Preferably, via conventional techniques, the inositol derivatives are purified.

When administered to a patient, e.g., a mammal for veterinary use or to a human for clinical use, or when made to contact a cell or tissue, the inositol derivatives can be used alone or in combination with any physiologically acceptable carrier or vehicle suitable for enteral or parenteral delivery. Where used for enteral, parenteral, topical, otic, ophthalmologic, intranasal, oral, sublingual, intramuscular, intravenous, subcutaneous, intravaginal, transdermal, or rectal administration, the physiologically acceptable carrier or vehicle should be sterile and suitable for in vivo use in a human, or for use in a veterinary clinical situation.

In addition, the inositol derivatives can be administered to patients or contacted with a cell or tissue in liposome formulations, which facilitate their passage through cell membranes. Accordingly, the relative impermeability of cell membranes to relatively polar inositol derivatives can be overcome by their encapsulation in liposomal formulations. The characteristics of liposomes can be manipulated by methods known to those of ordinary skill in the art, such that size, membrane fluidity, tissue targeting, and compound release kinetics are adapted to the particular condition (Georgiadis, NIPS 4:146 (1989)). Liposomes of various sizes and compositions that encapsulate the inositol derivatives for delivery can be achieved by methods known to those skilled in the art (See, for example, Hope et al., Biochem. Biophys. Acta 812:55 (1985); Hernandez, et al., J. Microencapsul. 4:315 (1987); Singh, et al., Cancer Lett. 84:15 (1994); and Dipali, et al., J. Pharm. Pharmacol. 48:1112 (1996)).

The inositol derivatives can be used in the form of a pharmaceutical preparation, for example, in solid, semisolid or liquid form, that contains at least one of the inositol derivatives of the present invention as a bioactive component, alone or in combination with an anti-inflammatory compound; in admixture with a carrier, vehicle or an excipient suitable for enteral or parental administration. Such anti-inflammatory compounds useful in this regard include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; and other anti-inflammatory agents including, but not limited to, colchicine, allopurinol, probenecid, sulfinpyrazone and benzbromarone.

In addition, the inositol derivatives of the present invention may be compounded, for example with a pharmaceutically acceptable carrier or vehicle for solid compositions such as tablets, pellets or capsules; capsules containing liquids; suppositories; solutions; emulsions; aerosols; sprays; suspensions or any other form suitable for use. Suitable carriers and vehicles include, for example, sterile water, sterile physiological saline, gum acacia, gelatin, starch paste, talc, keratin, colloidal silica, urea and the like. In addition, auxiliary, stabilizing, thickening, lubricating and coloring agents may be used. The inositol derivatives are present in the compositions in a therapeutically effective amount, i.e., an amount sufficient to restore normal mucosal secretions.

The compositions of this invention may be administered by a variety of methods including orally, sublingually, intranasally, intramuscularly, intravenously, subcutaneously, intravaginally, transdermally, rectally, by inhalation, or as a mouthwash in dosage unit formulations containing conventional nontoxic pharmaceutically acceptable carriers, adjuvants, and vehicles as desired. Topical administration may also involve the use of transdermal administration such as transdermal patches or ionophoresis devices. The preferred mode of administration is left to the discretion of the practitioner, and will depend in-part upon the desired site of action.

For example, when cystic fibrosis, chronic bronchitis or asthma affects the function of the lungs, the inositol derivatives can be administered as an atomized aerosol, via a nebulizer, or via perfusion in a fluorocarbon or synthetic pulmonary surfactant; alternatively, the inositol derivatives can be administered intravenously directly. Thus, the active compounds disclosed herein may be administered to the lungs of a patient by any suitable means, but are preferably administered by generating an aerosol comprised of respirable particles, the respirable particles comprised of the active compound, which particles the subject inhales. The respirable particles may be liquid or solid. The particles may optionally contain other therapeutic ingredients such as a sodium channel blocker as noted above, with the sodium channel blocker included in an amount effective to inhibit the reabsorption of water from airway mucous secretions. The particles may optionally contain other therapeutic ingredients such as antibiotics as described in U.S. Pat. Nos. 5,512,269 and 5,716,931 or Uridine Triphosphate Analogs as described in U.S. Pat. No. 5,292,498, nitric oxide inhibitors as described in U.S. Pat. No. 5,859,058, dinucleotides as described in U.S. Pat. No. 5,935,555, or organic acids as described in U.S. Pat. No. 5,908,611. Particles comprised of active compound for practicing the present invention should include particles of respirable size: that is, particles of a size sufficiently small to pass through the mouth and larynx upon inhalation and into the bronchi and alveoli of the lungs. In general, particles ranging from about 0.5 to 10 microns in size (more particularly, less than about 5 microns in size) are respirable. Particles of non-respirable size which are included in the aerosol tend to deposit in the throat and be swallowed, and the quantity of non-respirable particles in the aerosol is preferably minimized. For nasal administration, a particle size in the range of 10-500 μm is preferred to ensure retention in the nasal cavity.

Liquid pharmaceutical compositions of active compound for producing an aerosol can be prepared by combining the active compound with a suitable vehicle, such as sterile pyrogen free water. Other therapeutic compounds, such as a sodium channel blocker, may optionally be included. Solid particulate compositions containing respirable dry particles of micronized active compound may be prepared by grinding dry active compound with a mortar and pestle, and then passing the micronized composition through a 400 mesh screen to break up or separate out large agglomerates. A solid particulate composition comprised of the active compound may optionally contain a dispersant that serves to facilitate the formation of an aerosol. A suitable dispersant is lactose, which may be blended with the active compound in any suitable ratio (e.g., a 1 to 1 ratio by weight). Again, other therapeutic compounds may also be included.

The dosage of active compound for prophylaxis or treatment of lung disease will vary depending on the condition being treated and the state of the subject, but generally may be an amount sufficient to achieve dissolved concentrations of active compound on the airway surfaces of the subject of from about 10⁻⁹ to 10⁻³ Moles/liter, and more preferably from 10⁻⁷ to 10⁻⁵ Moles/liter. Depending on the solubility of the particular formulation of active compound administered, the daily dose may be divided among one or several unit dose administrations. Preferably, the daily dose is a single unit dose, which is preferably administered from 1 to 3 times a week. Treatments may continue week to week on a chronic basis as necessary (i.e., the active agent can be administered chronically). Administration of the active compounds may be carried out therapeutically (i.e., as a rescue treatment) or prophylactically, but preferably the compounds are administered prophylactically, either before substantial lung blockage due to retained mucus secretions has occurred, or at a time when such retained secretions have been at least in part removed, as discussed above.

Aerosols of liquid particles comprising the active compound may be produced by any suitable means, such as with a nebulizer. See, e.g., U.S. Pat. No. 4,501,729. Nebulizers are commercially available devices that transform solutions or suspensions of the active ingredient into a therapeutic aerosol mist either by means of acceleration of a compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. the carrier is typically water or a dilute aqueous alcoholic solution, preferably made isotonic with body fluids by the addition of, for example, sodium chloride. Optional additives include preservatives if the formulation is not prepared sterile, for example, methyl hydroxybenzoate, antioxidants, flavoring agents, volatile oils, buffering agents and surfactants. Aerosols of solid particles comprising the active compound may likewise be produced with any solid particulate medicament aerosol generator. Aerosol generators for administering solid particulate medicaments to a subject produce particles that are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation. A second type of illustrative aerosol generator comprises a metered dose inhaler. Metered dose inhalers are pressurized aerosol dispensers, typically containing a suspension or solution formulation of the active ingredient in a liquefied propellant. During use these devices discharge the formulation through a valve adapted to deliver a metered volume, typically from 10 to 150 μl, to produce a fine particle spray containing the active ingredient. Suitable propellants include certain chlorofluorocarbon compounds, for example, dichlorodifluorometiane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents. The aerosol, whether formed from solid or liquid particles, may be produced by the aerosol generator at a rate of from about 10 to 150 liters per minute, more preferably from about 30 to 150 liters per minute, and most preferably about 60 liters per minute. Aerosols containing greater amounts of medicament may be administered more rapidly.

Where the condition of the subject to be treated affects the gastrointestinal tract, the inositol derivatives can be administered rectally via enema or suppository, or orally in the form of a tablet or capsule formulated to prevent dissolution prior to entry into the afflicted portion of the gastrointestinal tract; when the cystic fibrosis affects vaginal secretions, the inositol derivatives can be administered intravaginally, in the form of a douche.

Compositions for oral delivery may be in the form of tablets, pills, troches, lozenges, aqueous or oily suspensions, granules or powders, emulsions, capsules, syrups or elixirs. Orally administered compositions may contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, compositions in tablet form may be coated to delay disintegration and absorption in the gastrointestinal tract thereby providing a sustained action over an extended period of time. Selectively permeable membranes surrounding an osmotically active driving compound are also suitable for orally administered compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Injectable preparations, for example, sterile injectable aqueous or oleagenous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in ⅓-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols, which are solid at ordinary temperatures but liquid at the rectal temperature and will therefore melt in the rectum and release the drug.

Aqueous suspensions containing the inositol derivatives may also contain one or more preservatives, such as, for example, ethyl or n-propyl-p-hydroxy-benzoate, one or more coloring agents, flavoring agents or sweetening agents.

Because the inositol derivatives are in the form of tetrakisphosphate, heptakis or octakis(acetoxymethyl or ethyl)esters, and because the inositol derivatives can contain —C₁-C₂₀ straight or branched chain alkyl, —OC(O)C₁-C₂₀ straight or branched chain alkyl or —OC₁-C₂₀ straight or branched chain alkyl groups, the inositol derivatives possess enhanced lipophilic properties which allow for passive diffusion across plasma membranes. This design permits the inositol derivatives to more easily penetrate cell membranes and travel to sites more easily and quickly.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W. (1976), p.33 et seq.

Without being bound by any particular theory, it is believed that the inositol derivatives function as “prodrugs” of a metabolized form of the inositol derivatives that are the actual pharmacological agent responsible for the modulation of sodium ion absorption. Such prodrugs, by virtue of their being more lipophilic than the actual pharmacological agents themselves, can more easily penetrate plasma membranes. Once within a secretory cell, the prodrugs are converted, generally enzymatically, to the active pharmacological agent. In addition, because in vivo conversion of a prodrug to its active pharmacological form generally occurs over a period of time, rather than instantaneously, the use of prodrugs offers the patient or subject the benefit of a sustained release of the pharmacological agent, generally resulting in a longer duration of action.

In addition, without being bound by any particular theory, it is believed that the inositol derivatives, by virtue of the fact that they comprise phosphate ester groups, are able to accumulate within “depots,” i.e., fatty domains of the brain, in particular, within cell membranes. Within in such depots, the inositol derivatives act to inhibit tissue damage caused by inflammation.

In a further embodiment, the present invention contemplates the use of an inositol derivative when delivered at a dose of about 0.001 mg/kg. to about 100 mg/kg body weight, preferably from about 0.01 to about 10 mg/kg body weight. The inositol derivatives can be delivered up to several times per day, as needed. Treatment can be continued, indefinitely to normalize mucosal hydration or sodium absorption or reduce excessive mucosal viscosity.

The amount of active ingredient that may be combined with the carrier materials to produce a single dosage form will vary depending upon the host treated and the particular mode of administration.

It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy.

While the compounds of the invention can be administered as the sole active pharmaceutical agent, they can also be used in combination with one or more other agents used in the treatment of the symptoms of cystic fibrosis, chronic bronchitis, asthma, inflammation and the like. For alleviating mucosal viscosity resulting from cystic fibrosis, a composition of the present invention may be administered that comprises an inositol derivative of the invention together with an agent useful for the treatment of inflammation-accompanying condition. For instance, for the treatment of cystic fibrosis, such an agent can be mucolytics (e.g., Pulmozyme® and Mucomyst®), purinergic receptor agonists such as uridine triphosphate (UTP), agents that suppress the cystic fibrosis transmembrane regulator (CFTR) premature stop mutation such as gentamycin, agents correcting the Delta F508 processing defect also known as “protein assist therapies” such as CPX™ (SciClone), Phenylbutyrate (Ucyclyd Pharma), INS365 (Insprie Pharmaceuticals), and genestein, and/or agents for the treatment of the accompanying infection such as tobramycin or aerosolized tobramycin (Tobi™), meropenem, RSV vaccine, IB605, Pa1806, anti-inflammatory agents such as DHA, rHEI, DMP777, IL10 (Tenovil) and/or agents triggering alternate chloride channels such as antibiotics such as Duramycin (Moli901-Molichem Medicines), or omeprazole, and/or purinergic agonists such as nucleotide or dinucleotide analogs, or agents affecting sodium transport such as amiloride, and/or agents affecting pH such as organic acids.

For the treatment of asthma, such agents can be corticosteroids—such as fluticasone propionate (Flovent®, Flovent Rotadisk®), budesonide (Pulmocort Turbuhaler®), flunisolide (Aerobid®), triamcinolone acetonide (Azmacort®), beclomethasone MDI (Beclovent®), antileukotrienes such as Zafirlukast (Accolate®, Zeneca®), Zileuton (Zyflo®), Montelukast or other therapies such as methotrexate, troleandomycin, gold, cyclosporine, 5′-lipoxygenase inhibitors, bronchodilators, or immunotherapeutic agents.

CPX is a caffeine-like compound being investigated by SciClone. In laboratory studies it appears to increase chloride secretion in CF tissues that have the delta F508 mutation, but not in tissues with other mutations or normal epithelial cells. It is unknown whether it would be effective in actual patients. Even if so, it would not benefit the 30% of CF sufferers who have other mutations.

Phenylbutyrate is a compound developed by Ucyclyd Pharma that targets the protein generated by the delta F508 mutation. The Cystic Fibrosis Foundation is currently sponsoring a Phase I clinical trial of the drug at the Johns Hopkins University. However, because high concentrations are necessary to be effective and the compound has an unappealing odor, other active analogs are currently being sought.

Duramycin is being developed by Molichem Medicines and forms pores in membranes allowing the passage of ions. However, it is difficult to regulate the concentration of the compound in the membrane and the efficacy of the compound.

Purinergic (P2Y2) receptor agonists such as adenosine triphosphate (ATP) and uridine triphosphate (UTP) stimulate calcium-dependent chloride channels (not CFTR channels). They are currently being investigated by researchers at the University of North Carolina (under the auspices of Inspire Pharmaceuticals, Inc.) and independently at Johns Hopkins University. Early trials indicate that this strategy could be useful in the treatment of cystic fibrosis and other chronic obstructive pulmonary disorders. However, the effectiveness of this approach may be limited by inflammation-related inhibitory signals.

The compounds of the invention may also be administered in combination with one or more sodium channel blockers. Sodium channel blockers which may be used in the present invention are typically pyrazine diuretics such as amiloride, as described in U.S. Pat. No. 4,501,729. The term “amiloride” as used herein includes the pharmaceutically acceptable salts thereof, such as (but not limited to) amiloride hydrochloride, as well as the free base of amiloride. The quantity of amiloride included may be an amount sufficient to achieve dissolved concentrations of amiloride on the airway surfaces of the subject of from about 10⁻⁷ to about 10⁻³ Moles/liter, and more preferably from about 10⁻⁶ to about 10⁻⁴ Moles/liter.

The methods of the present invention may also further comprise the step of removing retained mucus secretions from the lungs of the subject prior to the step of administering the active agent. This facilitates application of the active agent to the respiratory epithelia during the administering step. Such removal of retained mucus secretions can be carried out by any suitable means, including postural drainage, antibiotic administration (e.g., intravenous or inhalation administration of cephalosporin or aminoglycoside antibiotics such as Tobramycin), and/or inhalation administration of DNase. In addition, the present invention may be carried out on patients such as children prior to decline of respiratory function (e.g., patients essentially free of lung blockage due to retained mucus secretions). Such patients can be genetically predisposed to becoming afflicted with lung disease (e.g., cystic fibrosis) as hereinbefore described.

Alternatively, the compositions comprising an inositol derivative can be administered in combination with, prior to, concurrent with or subsequent to the administration of another agent useful for the treatment of cystic fibrosis accompanying condition, as described above.

In addition, the inositol derivatives can be used for research purposes; for example, to investigate the mechanism and activity of other agents thought to be useful for regulating mucosal hydration.

The foregoing may be better understood by reference to the following examples, which are provided for illustration and are not intended to limit the scope of the inventive concepts.

### EXAMPLE 1

Cystic Fibrosis Human Nasal Epithelial Cell Ussing Chamber Assay

Elevated basal I_{sc} measured in monolayer cultures in Ussing Chambers is a prominent characteristic of cultures of CF human nasal airway epithelia that distinguishes it from normal tissue. Although basal I_{sc} is largely driven by sodium channel activity (ENaC) it has been closely associated with both CFTR and Ca²⁺ activated Cl⁻ channels. (Devor and Pilewski, 1999; Inglis et al., 1999; Mall et al., 1999; Ramminger et al., 1999; Wang and Chan, 2000).

CF Human Nasal Epithelial (CFHNE) Cell Isolation and Proliferation: Nasal Polyps were surgically obtained from a CF patient at Children's Hospital (Seattle, Wash.), transported, on ice in a sterile container containing a 1:1 mixture of Dulbecco's modification of minimum essential medium Eagle and Ham's F-12 nutrient medium (DMEM/F-12)(Irvine Scientific, Santa Ana, Calif.) supplemented with 100 U/ml penicillin, 0.1 mg/ml streptomycin, 10 mM HEPES, and 2 mM L-glutamine. The tissue samples were aseptically removed from the transport medium and washed (repeated 5×) by suspending in 40 ml of Joklik's modification of minimum essential medium Eagle (JMEM) at 4° C., and centrifuging at 500 RPM. The supernatant was aspirated and discarded. The tissue was then transferred to JMEM containing 200 U/ml penicillin, 0.2 mg/ml streptomycin, 0.1 mg/ml gentamycin sulfate (Clonetics, San Diego, Calif.), and 0.1 μg/ml amphotericin-B (Clonetics), and 0.1% Protease (Sigma), washed an additional 2×, suspended in 15 ml in a 10 cm tissue culture dish, and incubated at 4° C. for 24 hours. The tissue samples were then gently triturated, the connective tissue aseptically removed, and the remaining cell suspension centrifuged at 1000 RPM for 5 min. The supernatant was aspirated and the pellet was resuspended in 10 ml JMEM with 0.025% trypsin-EDTA and allowed to incubate for 5 min. After 5 min., 10% Fetal Bovine Serum (FBS) was added to deactivate the trypsin, and the cell suspension was centrifuged at 1000 RPM. The supernatant was aspirated and the cell pellet was resuspended in a proliferation media consisting of Keratinocyte-Serurm Free Medium (KSFM)(Gibco-BRL, Grand Island, N.Y.) containing 5 ng/ml EGF (Gibco), 50 μg/ml BPE (Gibco), 100 U/ml penicillin, 0.1 mg/ml streptomycin, and 2 mM L-glutamine. The cell suspension was transferred to 2, 10 cm tissue culture dishes coated with 1 μg/cm² Vitrogen (Becton-Dickinson, Bedford, Mass.), incubated at 37° C. in a humidified atmosphere of 5% CO₂ and 95% air. The cells were allowed to grow for 6 days (70-80% confluence) with the media being replaced with fresh media every other day. The cells were then trypsinized using 0.025% trypsin-EDTA for 5 min. The cell suspension was collected, the trypsin deactivated with 10% FBS, and centrifuged at 1000 rpm for 5 min. The cells were then counted using a hemocytometer. There was a typical yield of 3×10⁶ cells per dish. The supernatant was aspirated and the cells were resuspended in KSFM and plated on 1 μg/cm² Vitrogen at a density of 5×10³ cells/cm².

CFHNE Cell Ussing Chamber Preparation: The CFHNE cells were prepared for Ussing Chamber studies using Snapwell permeable supports (0.4 μm pore size) (Corning Costar, Cambridge, Mass.) coated with 1 μg/cm² Vitrogen. Cells were plated at 10⁵ cells/cm² in KSFM. After 2 days, the media was changed to BEGM (a 1:1 mixture of DMEM (MediaTech/Cellgro, Hemdon, Va.) and BEBM (Clonetics/Biowhittaker, Walkersville, Md.), with the following supplements: hydrocortisone (0.5 μg/ml), insulin (5 μg/ml), transferrin (10 μg/ml), epinephrine (0.5 μg/ml), triiodothyronine (6.5 ng/ml), Bovine Pituitary Extract (52 μg/ml), EGF (0.5 ng/ml), all-trans retinoic acid (50 nM, Sigma), penicillin (100 U/ml, Sigma), streptomycin (0.1 mg/ml, Sigma), non-essential amino acids (1×, Sigma), and Bovine Serum Albumin (fatty acid-free, 3 μg/ml, Sigma).

Media supplements were from Clonetics unless otherwise indicated. The CFHNE cells were grown in the BEGM for 1 week, at which point an air-liquid interface (ALI) culture system was initiated. The cells were grown for an additional 2 weeks at ALI, being fed every other day basolaterally, until use in the Ussing chamber. Monolayers used for experiments were routinely fed the day before use.

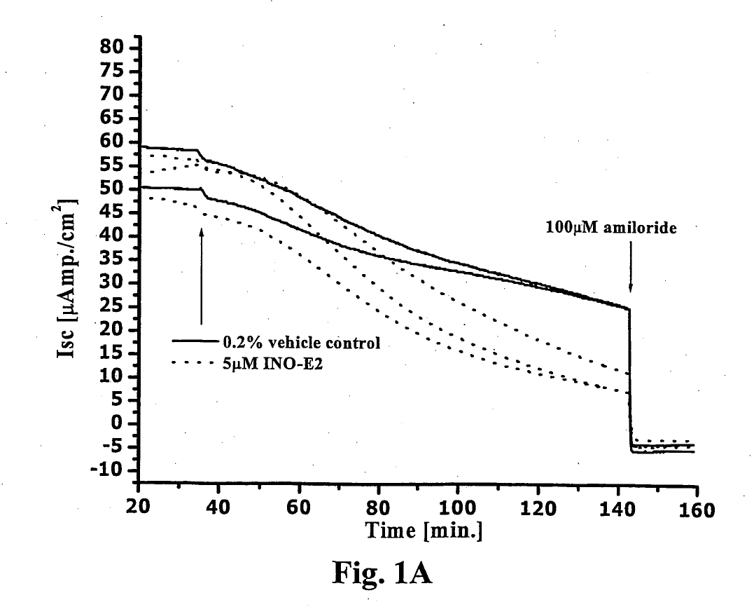

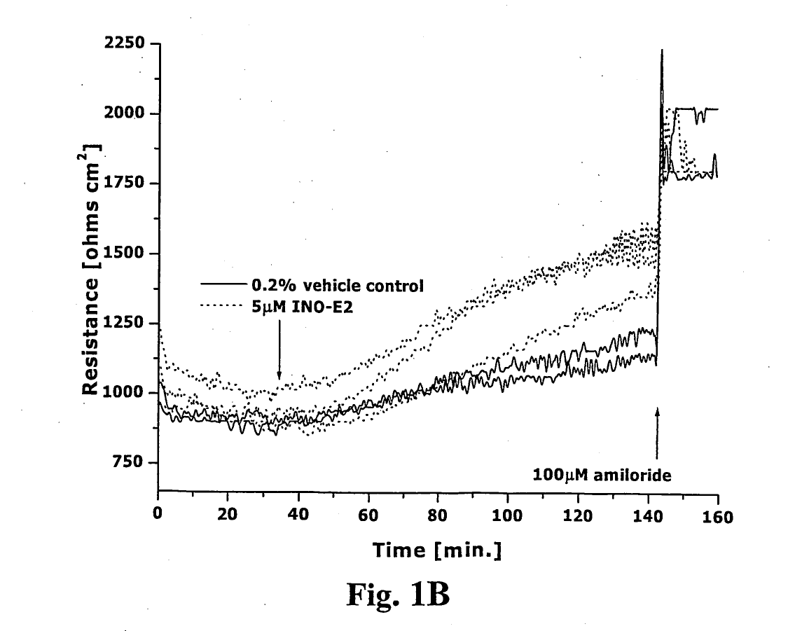

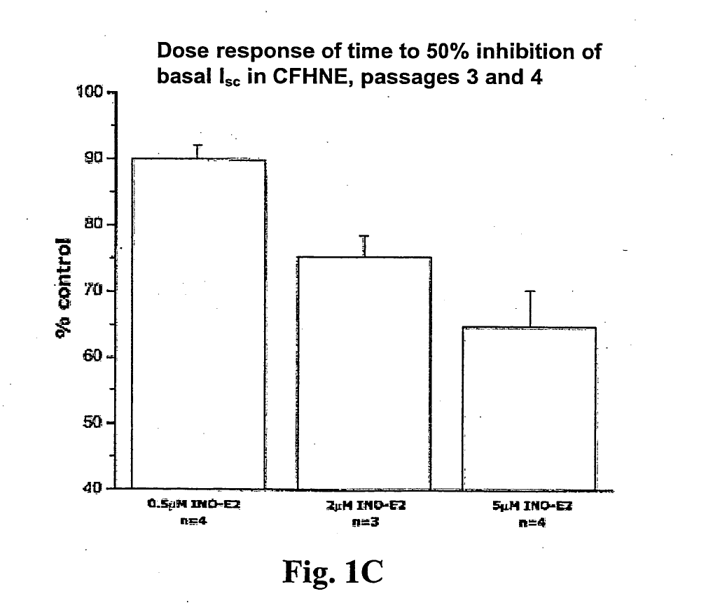

Acute Effects: Following the foregoing procedure, 2-O-butyryl-1-O-octyl-myo-inositol 3,4,5,6-tetrakisphosphate octakis (propionoxymethyl) ester (INO-E2) was shown to inhibit spontaneous amiloride-inhibitable Isc in reduced chloride and chloride free buffers. INO-E2 inhibits spontaneous amiloride-inhibitable Isc in a dose-dependent fashion (See FIG. 1A). This data demonstrates that INO-E2 has a direct effect on the apically located Na⁺ channel, ENaC. If the effect were secondary to an effect on a Cl⁻ conductance then it would not be possible to reproduce the observation in Cl⁻ free buffer. Thus, the effects of INO-E2 on basal amiloride-inhibitable Isc in primary CF human nasal epithelia were studied in Ussing chambers using low chloride and chloride free buffers. As can be seen in FIG. 1A, INO-E2 inhibition of spontaneous Isc is not reduced in Cl⁻ free buffer. This finding is demonstrates that INO-E2 has a direct action on ENaC that is distinct from its actions on calcium-activated Cl⁻ channels.

Long-term effects of INO-E2 on Basal Isc in CFHNE. The long-term effects of INO-E2 on basal spontaneous I_{sc} were studied or a variety of reasons:

1. Previous studies indicated that the onset of the effect was gradual and in some cases wasn't complete at the end of the experiment. This suggested a slowly building effect that might be long lasting.

2. A long lasting effect may make the drug attractive as a treatment for cystic fibrosis especially if this would mean that the patients only needed to take the drug once a day and obtained 24 hours of improved lung function. On the other hand a long lasting effect could also indicate toxicology issues that would need to be addressed.

3. In addition, delayed toxic effects could result in long-term damage of the monolayers that would not become apparent for several hours. Therefore, it is of interest to note whether the resistance or conductance was affected during this period. A reduction in resistance in monolayers treated with compound could indicate weakened tight junctions.

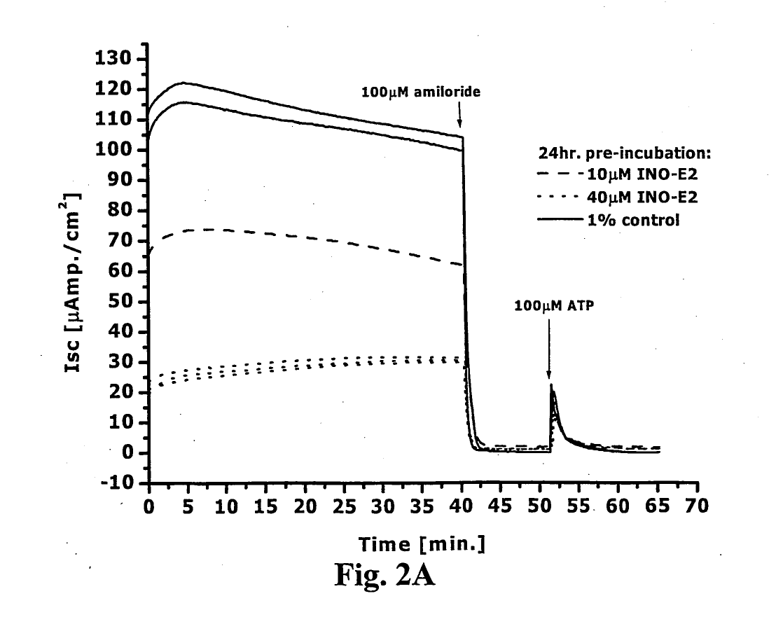

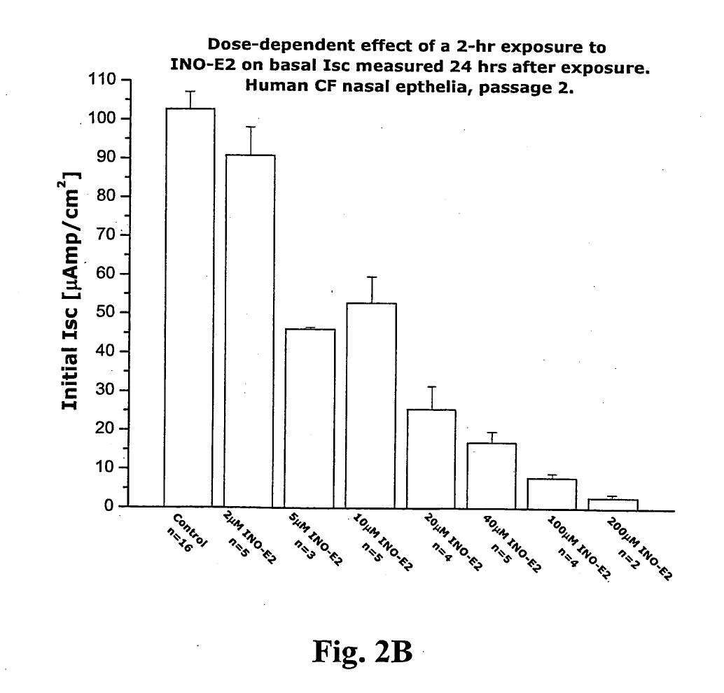

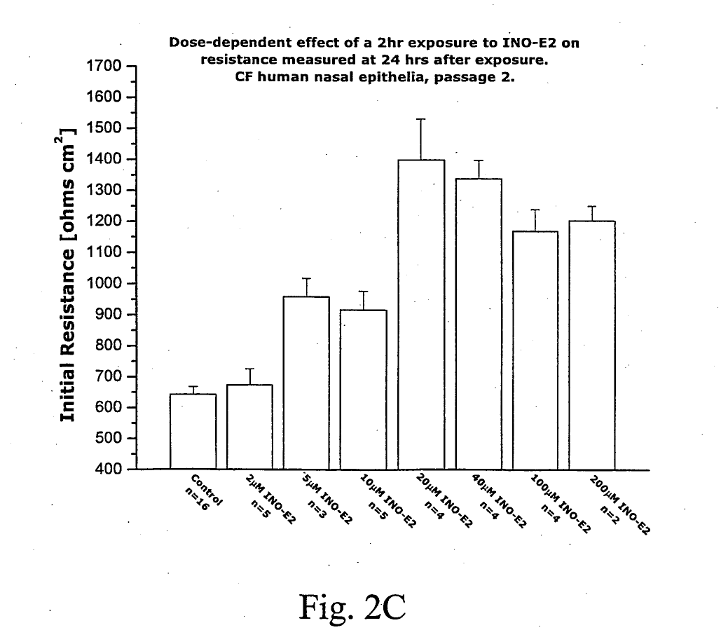

As can be seen in FIG. 2, INO-E2 administered 24 hrs prior to testing has a dose-dependent prolonged effect on basal amiloride-inhibitable I_{sc} in human nasal airway epithelia with an EC50 of approximately 5-10 μM. Monolayers were exposed to INO-E2 applied apically in Ringers for 2 hrs, washed, and then returned to air-liquid interface (ALI) for 24 hrs before the monolayers were mounted in Ussing Chambers for testing. This inhibition by INO-E2 is reversible since monolayers exposed to 5 μM INO-E2 for 2 hours and tested 48 hours later in Ussing chambers showed negligible inhibition of I_{sc}. As a control, the effect of pretreatment with phosphate-propionoxymethyl ester (26.6 μM) was tested using the same protocol. In this case there was no inhibition of I_{sc} indicating that the effect is not due to the protecting groups but the inositol polyphosphate analog itself. INO-E2 had no apparent toxic effects as evaluated by tight junction integrity since resistance (measured shortly after mounting in Ussing chambers) actually increased even with the highest concentration, 200 μM INO-E2.

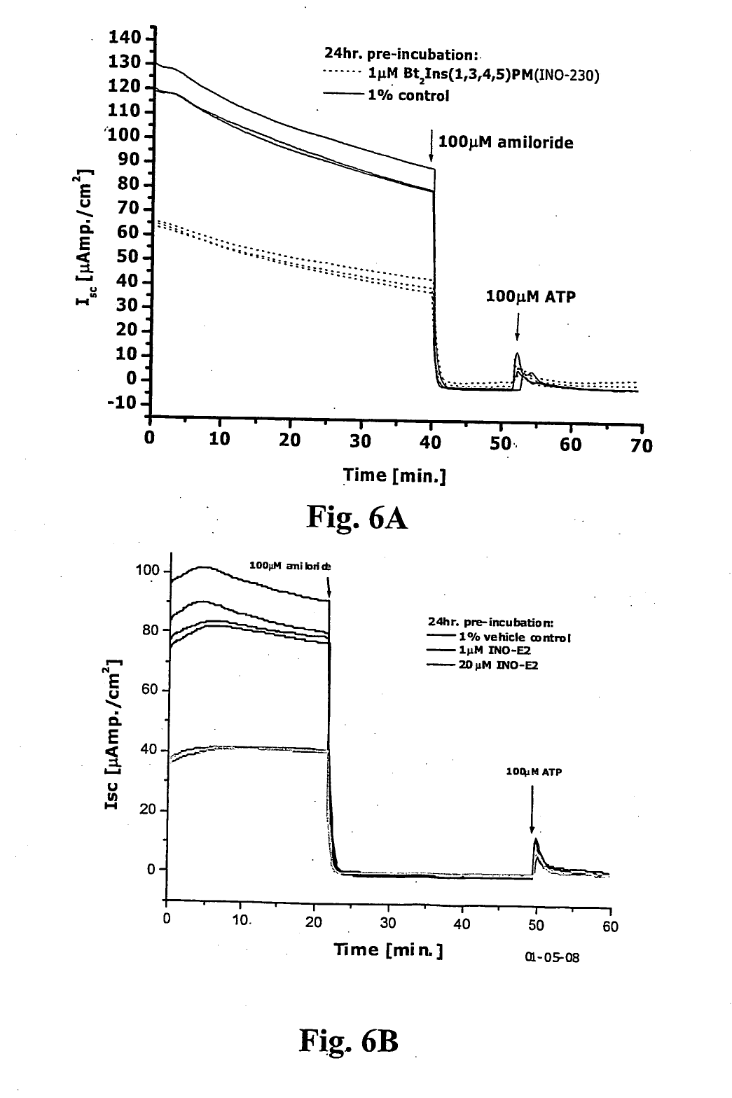

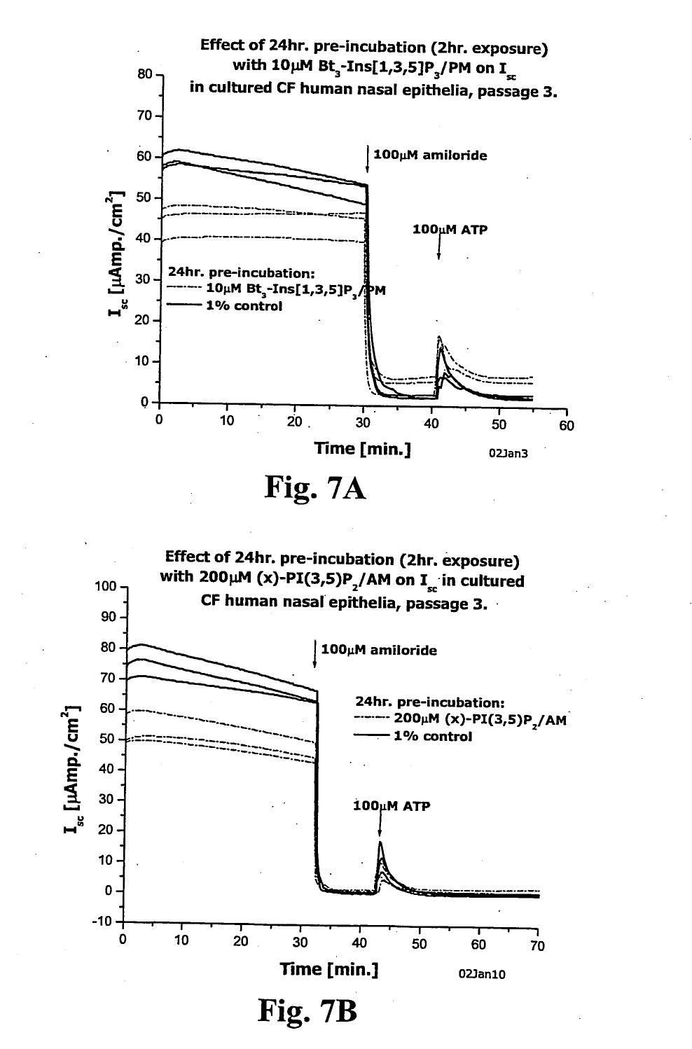

Using the foregoing procedure, the effect of 24 hr pre-incubation (2 hr. apical exposure) with 1 μM INO-230 (Bt₂Ins(1,3,4,5)P₄/PM was compared to INO-E2 (1-O-octyl-2-O-Bt-Ins(3,4,5,6)P₄/PM) on Isc in CFHNE monolayers. 1 μM INO-230 was applied apically for 2 hrs in sterile Ringer's buffer and then washed away. The air-liquid-interface was reestablished and the cells incubated for 22 hrs prior to measurement of short circuit current (Isc). The results are shown in FIG. 6A. As a control, preincubation consisted of 0.5% DMSO and 0.5% DMSO containing 5% Pluronic F-127 in Ringers buffer. For comparison, data obtained with INO-E2 under similar conditions are shown in FIG. 6B. Similarly, the effect of 24 hr pre-incubation (2 hr. apical exposure) with 1 μM Bt₃Ins(1,3,5)P₃/PM and phosphatidylinositol 3,5 bisphosphate (acetoxymethyl)ester are shown in FIGS. 7A and 7B, respectively.

### EXAMPLE 2

### Repeated Exposure to Low Doses of INO-E2

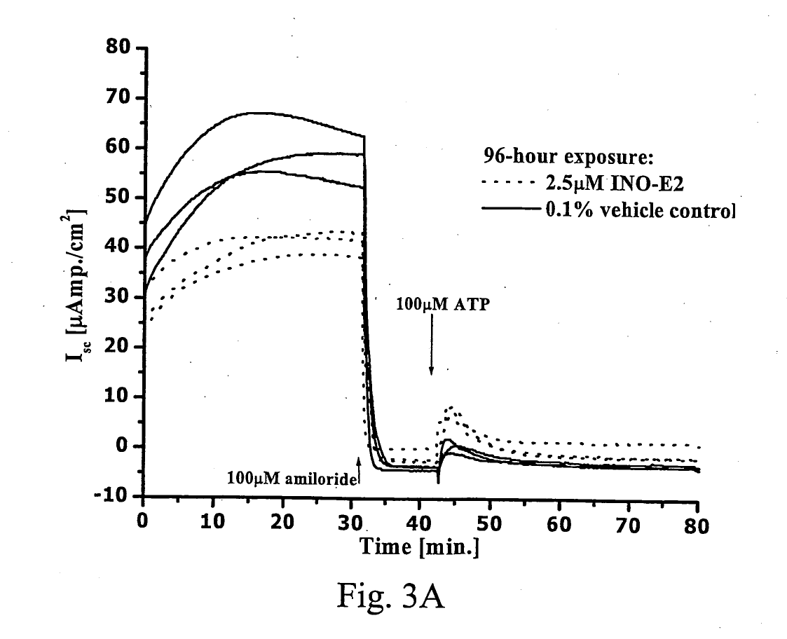

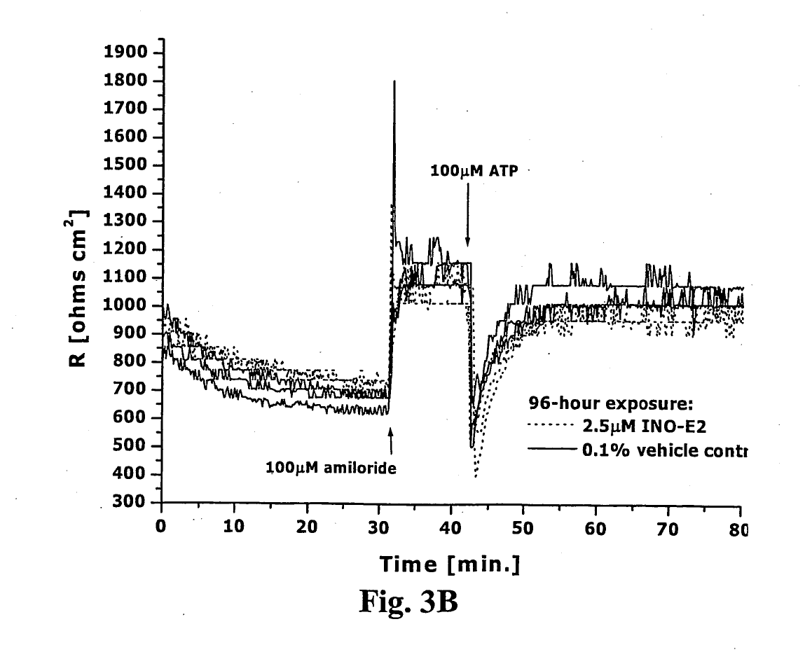

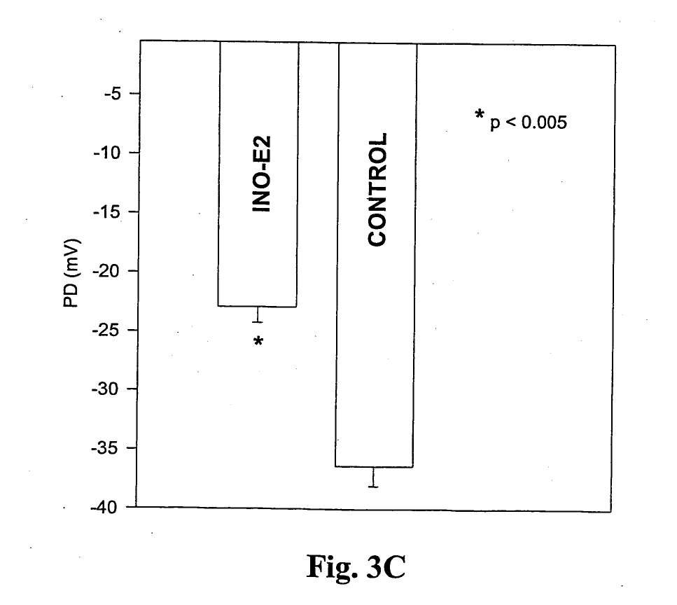

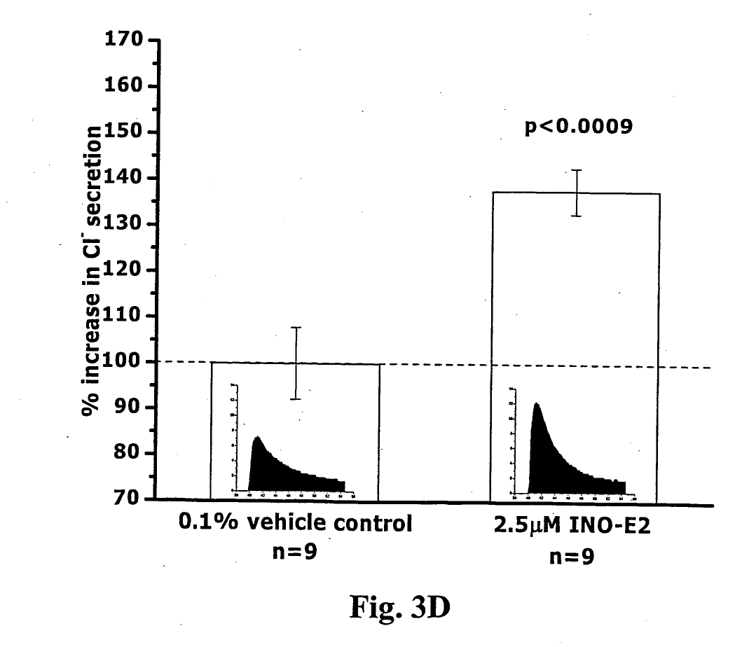

The therapeutic potential of INO-E2 was investigated by using protocols that model in vivo treatments. In this experiment the effects of repeated exposure to a low dose of INO-E2 (2.5 μM) on basal I_{sc} and responsiveness to calcium-mobilizing agonists (ATP) in human nasal CF epithelia (CFHNE) was tested. While in previous experiments 2.5 μM INO-E2 had an acute inhibitory effect on I_{sc}, this effect had dissipated after 24 hrs. T here was no apparent difference in responses to compound added to the apical or basolateral compartment. However, when added repeatedly, there is a cumulative beneficial effect with no indication of toxicity, as shown in FIG. 3. This series of experiments provides information that useful for deriving the optimal therapeutic dose and delivery schedule for in vivo tests.

INO-E2 Stimulates a CL⁻ Current in Whole Cell Patch Clamp Mode.

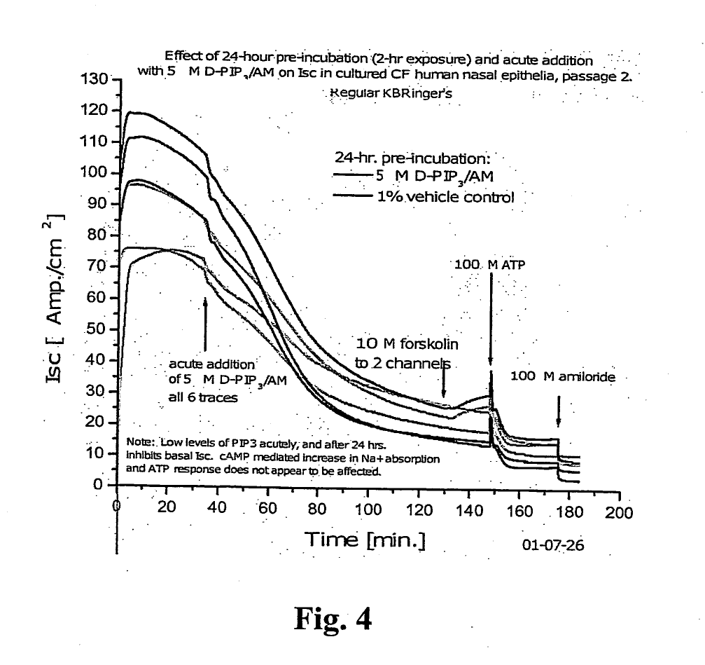

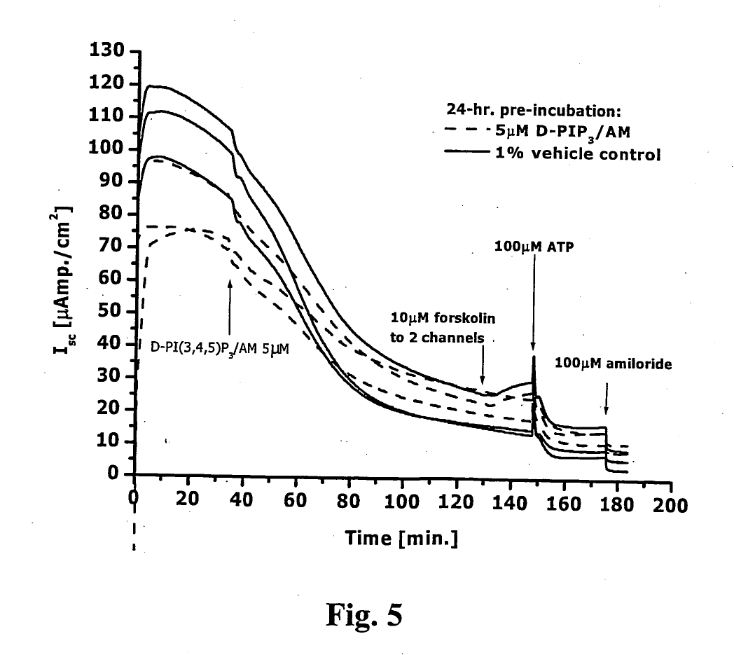

As can be seen in FIG. 4 Cl⁻ currents can be observed in perforated patch whole cell recordings in CFHNE cell within 5-15 minutes following 10 μM INO-E2 addition. In FIGS. 4A and 4B, the response to 10 μM INO-E2 is contrasted with the response to ATP. ATP elicited no further increase in whole cell chloride current suggesting that ATP and INO-E2 are stimulating the same chloride channel. A low to intermediate increase in chloride current in other perforated patch recordings was obtained with 5 and 10 μM INO-E2 (data not shown). In FIGS. 4C and 4D, the calcium-activated Cl⁻ channel inhibitor niflumic acid (100 μM) completely blocked the INO-E2 mediated whole cell current providing further evidence that INO-E2 is stimulating a calcium-activated Cl⁻ channel.

These data indicate that INO-E2, similar to other agents that stimulate Ca²⁺-activated Cl⁻ channels such as purinergic agonists, modulates both Cl⁻ and Na⁺ conductances.

INO-E2 is effective for prolonged periods in reducing basal spontaneous I_{sc}. The experiments were designed to simulate some of the circumstances of drug delivery where compound would be present in the airways for a discrete period following administration of compound. In the 24 hr preincubation experiments, the EC₅₀ for the chronic administration of compound after 24 hrs is between 5 and 10 μM or between 50-80% decreased from the potency observed following acute addition of compound where the EC₅₀ is approximately 1-2 μM (data not shown).

On the other hand, responses to repeated exposure of 1-2.5 μM INO-E2 over 96 hours demonstrate that repeated exposure to low concentrations of INO-E2 reverses the pathologically elevated basal I_{sc} in CF airway tissue and augments subsequent stimulation of Ca²⁺ activated Cl⁻ secretion through other pathways. No evidence of toxicity such as deterioration of tight junctions was observed. In fact, control basal resistance values were close to the resistance values seen in treated monolayers, with the slight increase in resistance presumably attributed to the reduction in transepithelial Na⁺ movement. This data suggests that it may be possible to achieve desired therapeutic results by administering the drug every other day or every third day. In addition, concentrations of compound which are in 100 fold excess of the EC₅₀ observed following acute addition was still effective with no decrement in the integrity of the monolayers. In fact, the resistance of the monolayers was increased. This suggests that the compounds are not cytotoxic at therapeutic concentrations.

Furthermore, responses to ATP, a calcium elevating agonist were elevated in the INO-E2 treated monolayers. These data also indicate that in addition to its “stand alone” effects, INO-E2 is useful as an adjunctive treatment in combination with other calcium elevating drugs in the treatment of CF airway disease.

### EXAMPLE 3

### Blue Dextran Volume Transport Assay

In normal human airway epithelia, Na⁺ and Cl⁻ currents (CFTR and Ca²⁺-activated Cl⁻ currents) contribute to airway surface liquid (ASL) fluid volume regulation depending on signaling equilibria. In contrast, in human CF airway epithelia, Na⁺ currents through ENaC dominate basal ASL volume regulation accompanied by a relatively minor contribution through Ca²⁺-activated Cl⁻ currents. The combination of enhanced ENaC currents and transient Ca²⁺-activated Cl⁻ currents in CF result in an inadequate hydration of the ASL and reduction of mucociliary clearance. Since INO-E2 reduces Na⁺ transport through ENaC, we hypothesized that INO-E2 would have a functional effect, similar to amiloride, and inhibit fluid absorption. To test this hypothesis, we exposed well differentiated monolayer cultures of CF nasal epithelia to an apically applied buffer containing INO-E2 and a known concentration of the non-permeable molecule Blue Dextran (BD). The resulting reduction in the ability of these monolayers to concentrate BD was taken as a functional indicator of INO-E2's involvement in the inhibition of ENaC.

HEPES Modified Ringer's Buffer (HMRB) was used for these assays. HMRB was sterilized by positive pressure filtration through a 0.2 um syringe tip filter (Gelman acrodisc). The BD stock solution was aseptically prepared with sterile buffer (2 mg BD/ml buffer). Compounds to be tested were solubilized in sterile HMRB containing 1 μM BD (final concentration of vehicle is 0.1% (1:1, DMSO+DMSO containing 5% pluronic-F127). The composition of HMRB (pH 7.3) is as follows (in mM): 135 NaCl, 1.2 CaCl₂, 1.2 MgCl₂, 2.4 K₂HPO₄, 0.6 KH₂PO₄, 10 HEPES, 10 glucose. 2001 μl of the solution was placed on the apical surface of monolayers grown on Corning-Costar snapwells or transwells (1.12 cm²) and placed in a humidified incubator for 18 hrs. After 18 hours, 60 μl of the remaining apical buffer was sampled and transferred to a 0.7 ml microcentrifuge tube for later analysis. A standard concentration curve was obtained by determining the optical densities of a serial dilution of 10 μM Blue Dextran in HMRB placed in a Falcon 3027 96-well plate. The optical density of BD in the solution was obtained by analysis at 660 nm in a Packard Spectracount. Standards and samples were analyzed on the same plate. 50 μl volumes were used for all samples and standards. The [BD] of the samples was determined by extrapolation from the standard curve using the Packard I-Smart software. The increase in [BD] from the starting value of 1 μM was taken to be an indication of the magnitude of volume absorption occurring across the monolayer. The rate of absorption was calculated by dividing the change in volume by the duration of the experiment. This value was normalized to a surface area of 1 cm², to give μl·cm²⁻¹·hr⁻¹. To address evaporative loss, which would appear in the data as artificially high absorptive rates, dummy membranes were constructed by attaching a thin Sylgard sheet to snapwells/transwells with silicon sealant. These dummy membranes were used in parallel with the epithelial monolayers to assess evaporative loss for each experiment. It was thus determined that evaporative loss did not contribute to the calculated absorption rate.

Amiloride Dose-Dependently Inhibits Fluid Absorption Using the Blue Dextran Assay.

The basal absorption rate using the Blue Dextran Assay ranged between 4 and 6 μl/cm², consistent with values for CF tissue reported in the literature (Jiang, et al., 1993 Science, 262 p424-427). To assess the ability of the Blue Dextran Assay to measure relevant changes in fluid secretion, the effect of the sodium channel blocker amiloride was tested. A large component of the abnormal fluid absorption in CF is due to accelerated sodium absorption and blocking the sodium channel with amiloride would be expected to significantly reduce fluid absorption. As expected, amiloride inhibited fluid absorption measured by the Blue Dextran assay in a dose dependent fashion. Therefore, this assay is suitable for evaluating the therapeutic potential of other compounds that inhibit sodium channels, such as inositol polyphosphate analogs.

The following compounds were tested in accordance with the foregoing procedure:

TABLE 1
Test Compounds

| Com- | | |
|---|---|---|
| pound | | |
| ID No. | Compound | FIG. |
| 4981 | 1,2,5-tri-O-butylyl-myo-inositol 3,4,6-trisphosphate | 8 |
| | hexakis (propionoxymethyl) ester (ent-TMX/PM) | |
| 4982 | 2,3,5-tri-O-butyryl-myo-inositol 1,4,6-trisphosphate | 9 |
| | hexakis (propionoxymethyl) ester (TMX/PM) | |
| 4984 | 2,3-camphanylidene-myo-inositol 1,4,5,6- | 10 |
| | tetrakisphosphate octakis (propionoxymethyl) ester | |
| 4992 | 2,4,6-tri-O-butyryl-myo-inositol 1,3,5-trisphosphate | 11 |
| | hexakis (propionoxymethyl) ester | |
| 4987 | 2-O-butyryl-3-O-octyl-myo-inositol 1,4,5,6- | 12 |
| | tetrakisphosphate octakis (propionoxymethyl) ester | |
| | (INO-E3) | |
| 4995 | 2-O-butyryl-1-O-octyl-myo-inositol 3,4,5,6- | 13 |
| | tetrakisphosphate octakis (propionoxymethyl) ester | |
| | (INO-E2) | |
| 4997 | 2-O-butyryl-1-O-(3-phenylpropyl)-myo-inositol 3,4,5,6- | 14 |
| | tetrakisphosphate octakis (propionoxymethyl) ester | |
| 4991 | 2,6-Di-O-butyryl-myo-inositol 1,3,4,5-tetrakisphosphate | 15 |
| | octakis (propionoxymethyl) ester | |
| 4996 | 1,2-camphanylidene-myo-inositol 3,4,5,6- | 16 |
| | tetrakisphosphate octakis (propionoxymethyl) ester | |

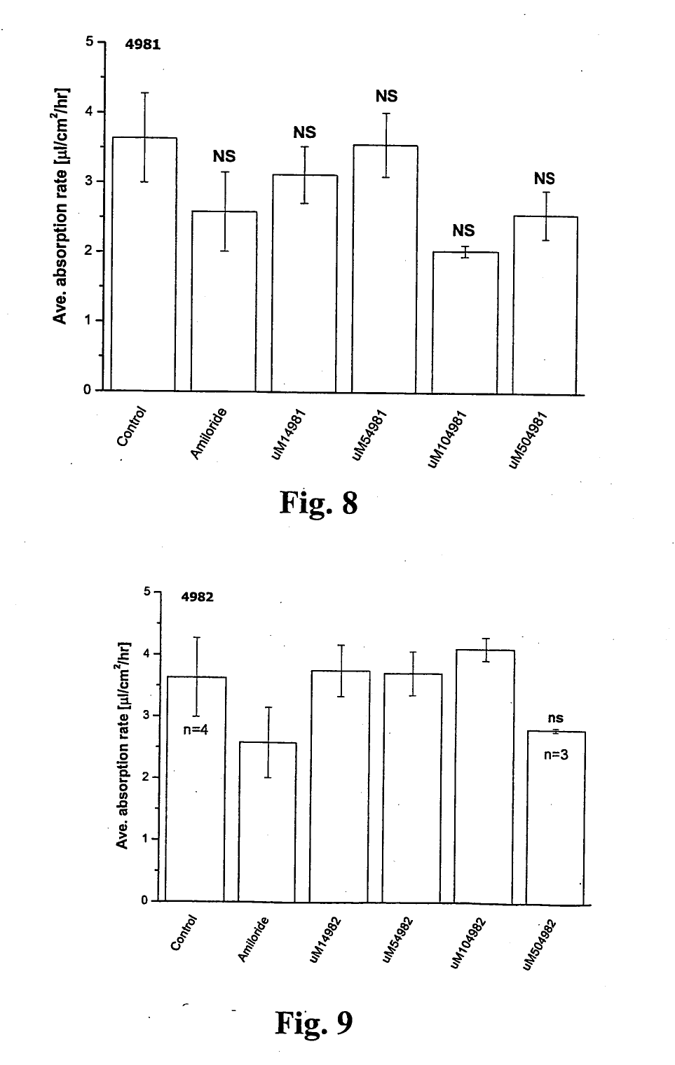

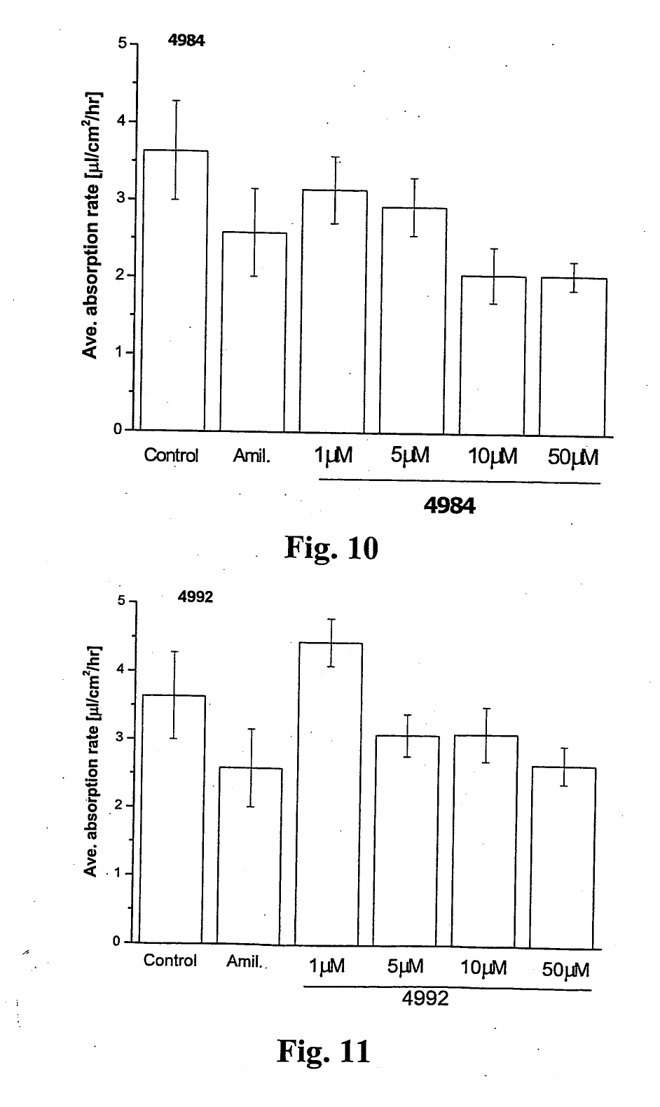

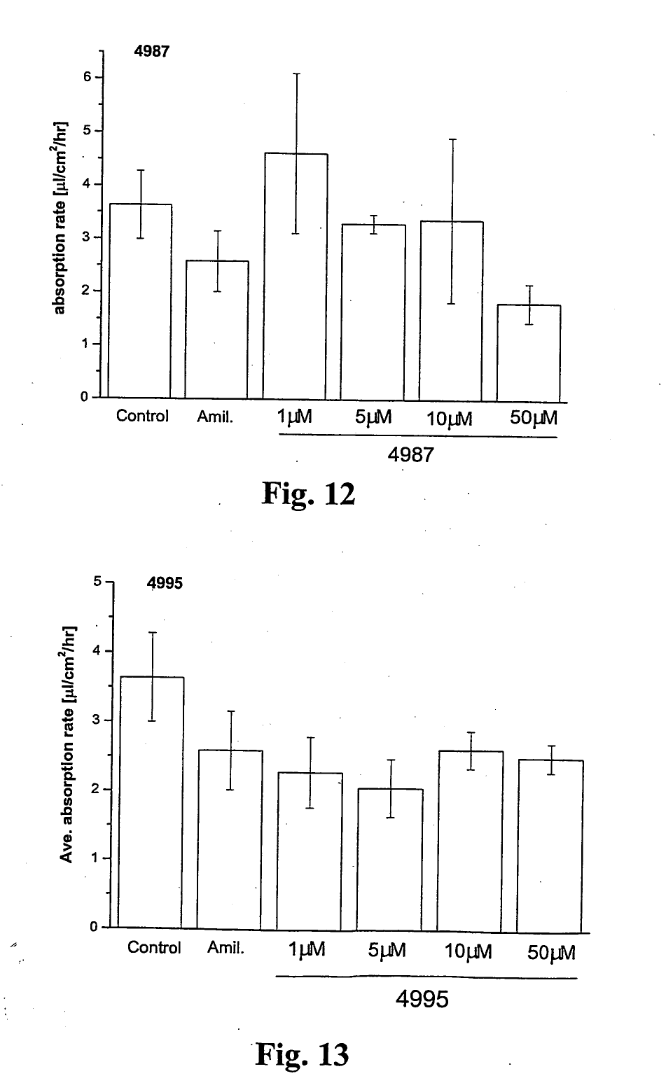

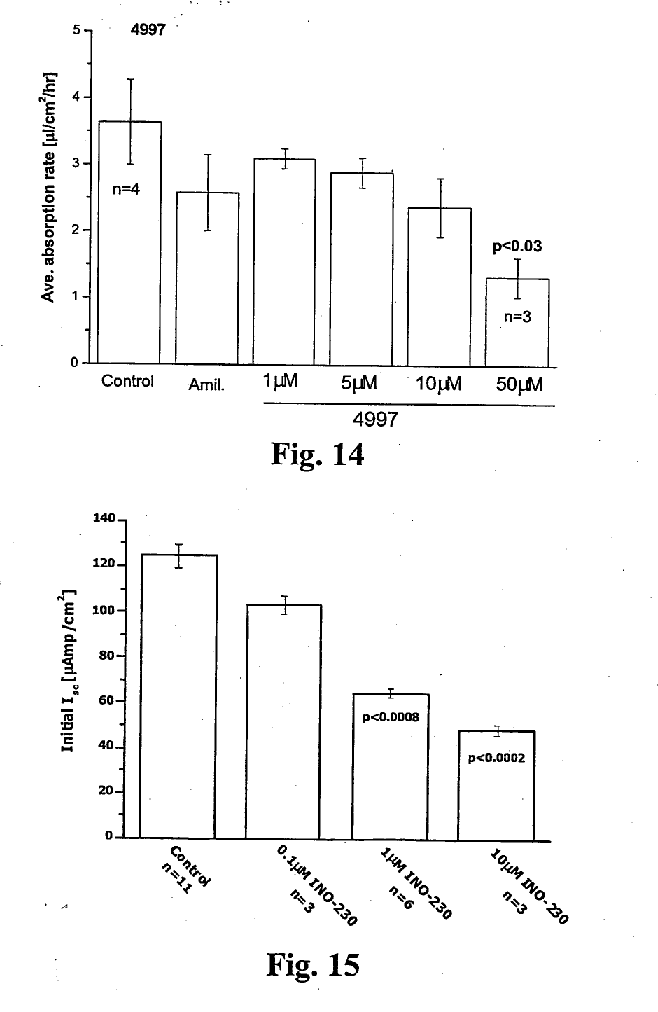

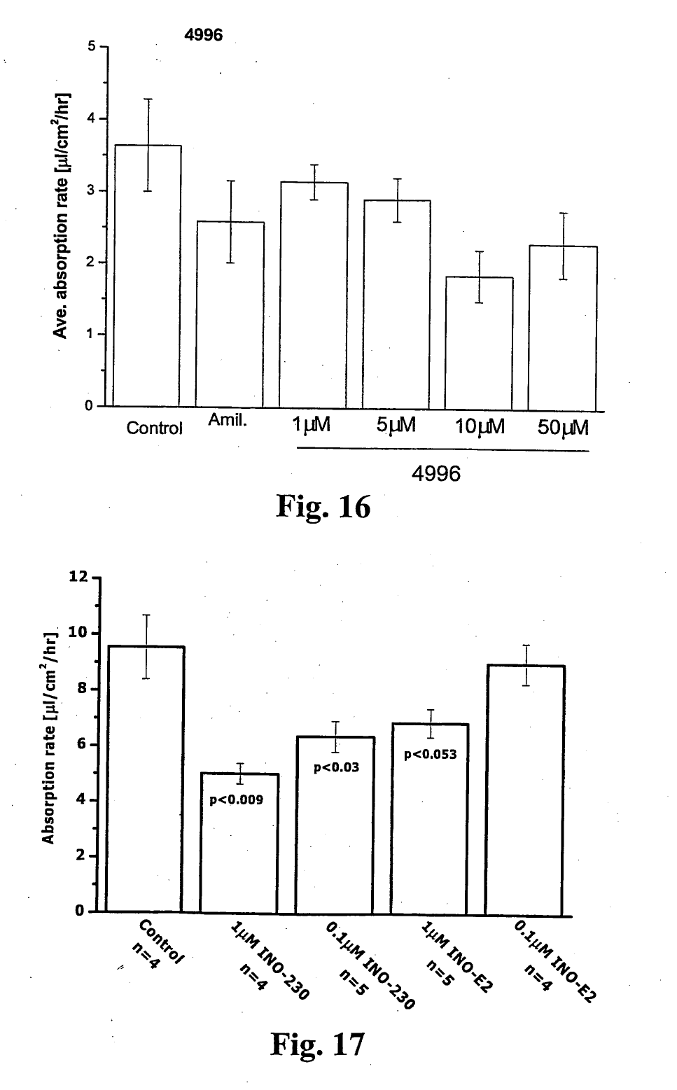

The dose response analysis of the effects of a series of inositol polyphosphate analogs on fluid absorption in human CF nasal epithelia using the blue dextran (BD) assay are shown in FIGS. 8-17. In these Figures, rates are compared with absorption rates in the presence of amiloride. Data are shown as means+/−SEM in the bar graphs of FIGS. 8-17.

These data show the effects of the tested inositol polyphosphate analogs on the inhibition of the average fluid absorption rate in human CF nasal airway epithelia. For comparison, 100 μM amiloride, a ligand that binds the apical sodium channel (ENaC) acutely inhibits fluid absorption, was included for comparison. INO-E2 (INO-4995) is as potent as amiloride in a fluid secretion assay. Its effects are longer-lasting under conditions that model therapeutic exposure times and repeated exposure to micromolar levels of INO-E2 results in corrective changes in basal I_{sc} and responses to calcium secretagogues. Extrapolating from clinical studies with aerosolized amiloride formulations, therapeutically effective concentrations of INO-E2 are achievable in vivo using currently available formulation strategies.

The data indicate that while all analogs shown above modulated fluid absorption, certain compounds were more potent than others. For instance, 5 uM 4992 was nearly as potent as 100 μM amiloride in this assay. While 1 μM 4997 had 50% of the activity of 100 μM amiloride, 100 μM 4984 was more potent than 100 μM amiloride. These data demonstrate the efficacy of inositol polyphosphate analogs in modulating a major consequence of abnormal sodium channel regulation in CF epithelia, enhanced fluid absorption.

### EXAMPLE 4

### Repeated Exposure to Low Concentrations in the Blue Dextran Volume Transport Assay

Drugs administered via nebulizer to patients have a relatively short residence time due to expulsion and/or hydrolysis. This relatively brief period of drug exposure could account for the short term effects obtained with extracellularly acting agents such as amiloride or purinergic agonists. In contrast, membrane permeant analogs of inositol polyphosphates are not subject to those factors once taken up into the mucosal epithelia. Therefore it is of interest to test the duration of the effect on fluid absorption following pulsed exposure to compounds. We compared the long-term effect of pulsed exposure to INO-E2 and Bt₂Ins(1,3,4,5)P₄/PM (INO-230, sic INO-4991) (FIG. 17) to that of amiloride. The data show that INO-E2 and INO-230 both exert prolonged effects on fluid secretion following a brief exposure. INO-230 appears to be more potent than INO-E2. 10 μM INO-230 significantly inhibits fluid absorption measured 42 hrs after a 2 hr exposure.

Following the procedure of Example 3, repeated exposure of the apical surface of monolayers to low doses Bt₂Ins(1,3,4,5)P₄/PM (INO-230) over a period of 96-192 hours extends potency of inhibition of fluid absorption into the nanomolar range, as shown in FIG. 17.

### REFERENCES

Bennett, W. D., Olivier, K N., Zeman, K. L., Hohneker, K. W., Boucher, R. C., and Knowles, M. R. (1996). Effect of uridine 5′-triphosphate plus amiloride on mucociliary clearance in adult cystic fibrosis. Am J Respir Crit Care Med 153, 1796-801.
Blank, U., C. Ruckes, W. Clauss, T. Hofmann, H. Lindemann, G. Munker, and W. Weber. Cystic fibrosis and non-cystic-fibrosis human nasal epithelium show analogous Na+ absorption and reversible block by phenamil. Pflugers Arch 434: 19-24., 1997.
Boucher, R. C., Stutts, M. J., Knowles, M. R., Cantley, L., and Gatzy, J. T. (1986). Na+ transport in cystic fibrosis respiratory epithelia. Abnormal basal rate and response to adenylate cyclase activation. J Clin Invest 78, 1245-52.
Carew, M. A., and Thorn, P. (2000). Carbachol-stimulated chloride secretion in mouse colon: evidence of a role for autocrine prostaglandin E2 release. Exp Physiol 85, 67-72.
Carew, M. A., Yang, X., Schultz, C., and Shears, S. B. (2000). Ins(3,4,5,6)P{sub4} inhibits an apical calcium-activated chloride conductance in polarized monolayers of a cystic fibrosis cell-line. J Biol Chem.
Clarke, L. L., Grubb, B. R., Yankaskas, J. R., Cotton, C. U., McKenzie, A., and Boucher, R. C. (1994). Relationship of a non-cystic fibrosis transmembrane conductance regulator-mediated chloride conductance to organ-level disease in Cftr(−/−) mice. Proc Natl Acad Sci USA 91, 479-83.
Devor, D. C., and Pilewski, J. M. (1999). UTP inhibits Na+ absorption in wild-type and DeltaF508 CFTR-expressing human bronchial epithelia Am J Physiol 276, C827-37.
Egan, M., Flotte, T., Afione, S., Solow, R., Zeitlin, P. L., Carter, B. J., and Guggino, W. B. (1992). Defective regulation of outwardly rectifying Cl− channels by protein kinase A corrected by insertion of CFTR [see comments]. Nature 358, 581-4.
Gabriel, S. E., Clarke, L. L., Boucher, R. C., and Stutts, M. J. (1993). CFTR and outward rectifying chloride channels are distinct proteins with a regulatory relationship. Nature 363, 263-8.
Greger, R (2000). Role of CFTR in the colon. Annu Rev Physiol 62, 467-91.
Grubb, B. R., Paradiso, A. M., and Boucher, R. C. (1994). Anomalies in ion transport in CF mouse tracheal epithelium. Am J Physiol 267, C293-300.
Ho, M. W., Shears, S. B., Bruzik, K. S., Duszyk, M., and French, A. S. (1997). Ins(3,4,5,6)P₄ specifically inhibits a receptor-mediated Ca2+-dependent Cl− current in CFPAC-1 cells. Am J Physiol 272, C1160-8.
Inglis, S. K., Collett, A., McAlroy, H. L., Wilson, S. M., and Olver, R. E. (1999). Effect of luminal nucleotides on Cl− secretion and Na+ absorption in distal bronchi. Pflugers Arch 438, 621-7.
Ismailov I I, Fuller C M, Berdiev B K, Shlyonsky V G, Benos D J, Barrett K E (1996) A biologic function for an “orphan” messenger: D-myo-inositol 3,4,5,6-tetrakisphosphate selectively blocks epithelial calcium-activated chloride channels. Proc Natl Acad Sci U S A 17;93(19):10505-9.
Knowles, M. R., Stutts, M. J., Yankaskas, J. R, Gatzy, J. T., and Boucher, R. C., Jr. (1986). Abnormal respiratory epithelial ion transport in cystic fibrosis. Clin Chest Med 7,285-97.
Konig, J., R. Schreiber, T. Voeloker, M. Mall, and K. Kunzelmann. The cystic fibrosis transmembrane conductance regulator (CFTR) inhibits ENaC through an increase in the intracellular Cl− concentration. EMBO Rep 2: 1047-51., 2001. Leung, A. Y., Wong, P. Y., Gabriel, S. E., Yankaskas, J. R., and Boucher, R. C. (1995). cAmp- but not Ca(2+)-regulated Cl⁻ conductance in the oviduct is defective in mouse model of cystic fibrosis. Am J Physiol 268, C708-12.
Mall, M., Bleich, M., Kuehr, J., Brandis, M., Greger, R., and Kunzelmann, K (1999). CFTR-mediated inhibition of epithelial Na+ conductance in human colon is defective in cystic fibrosis. Am J Physiol 277, G709-16.
Matsui, H., Davis, C. W., Tarran, R., and Boucher, R. C. (2000). Osmotic water permeabilities of cultured, well-differentiated normal and cystic fibrosis airway epithelia. J Clin Invest 105, 1419-27.
Matsui, H., Grubb, B. R., Tarran, R., Randell, S. H., Gatzy, J. T., Davis, C. W., and Boucher, R. C. (1998). Evidence for periciliary liquid layer depletion, not abnormal ion composition, in the pathogenesis of cystic fibrosis airways disease. Cell 95, 1005-15.
Nagel, G., T. Szellas, J. R. Riordan, T. Friedrich, and K. Hartung. Non-specific activation of the epithelial sodium channel by the CFTR chloride channel. EMBO Rep 2: 249-54., 2001. Pilewski, J. M., and Frizzell, R. A. (1999). Role of CFTR in airway disease. Physiol Rev 79, S215-55.

Ramminger, S. J., Collett, A., Baines, D. L., Murphie, H., McAlroy, H. L., Olver, R. E., Inglis, S. K., and Wilson, S. M. (1999). P2Y2 receptor-mediated inhibition of ion transport in distal lung epithelial cells. Br J Pharmacol 128, 293-300.
Rozrnahel, R., Wilschanski, M., Matin, A., Plyte, S., Oliver, M., Auerbach, W., Moore, A., Forstner, J., Durie, P., Nadeau, J., Bear, C., and Tsui, L. C. (1996). Modulation of disease severity in cystic fibrosis transmembrane conductance regulator deficient mice by a secondary genetic factor [published erratum appears in Nat Genet 1996 May;13(1):129]. Nat Genet 12, 280-7.
Schwiebert, E. M., Egan, M. E., Hwang, T. H., Fulmer, S. B., Allen, S. S., Cutting, G. R., and Guggino, W. B. (1995). CFTR regulates outwardly rectifying chloride channels through an autocrine mechanism involving ATP. Cell 81, 1063-73.
Vajanaphanich, M., Schultz, C., Rudolf M. T., Wasserman, M., Enyedi, P., Craxton, A., Shears, S. B., Tsien, R. Y., Barrett, K. E., and Traynor-Kaplan, A. (1994). Long-term uncouplng of chloride secretion from intracellular calcium levels by Ins(3,4,5,6)P₄. Nature 371, 711-4.
Veeze, H. J., Halley, D. J., Bijman, J., de Jongste, J. C., de Jonge, H. R., and Sinaasappel, M. (1994). Determinants of mild clinical symptoms in cystic fibrosis patients. Residual chloride secretion measured in rectal biopsies in relation to the genotype. J Clin Invest 93, 461-6.
Wang, X. F., and Chan, H. C. (2000). Adenosine triphosphate induces inhibition of Na(+) absorption in mouse endometrial epithelium: a Ca(2+)-dependent mechanism. Biol Reprod 63, 1918-24.
Xie, W., Solomons, K. R., Freeman, S., Kaetzel, M. A., Bruzik, K. S., Nelson, D. J., and Shears, S. B. (1998). Regulation of Ca2+-dependent Cl− conductance in a human colonic epithelial cell line (T84): cross-talk between Ins(3,4,5,6)P₄ and protein phosphatases. J Physiol (Lond) 510, 661-73.

While the preferred embodiments of the invention have been illustrated and described, it will be appreciated that various changes can be made therein without departing from the spirit and scope of the invention.

1. A method for modulating sodium ion absorption by epithelial cells, comprising treating the cells with an effective amount of a sodium uptake modulating inositol polyphosphate compound.

2. A method for modulating sodium ion absorption by epithelial cells in a human or animal patient in need of such treatment, comprising administering to the patient a therapeutically effective amount of a sodium uptake modulating inositol polyphosphate compound.

3. The method of claim 2, wherein the sodium uptake modulating inositol polyphosphate compound is a sodium uptake inhibiting inositol polyphosphate compound.

4. The method of claim 2, wherein the sodium uptake inhibiting inositol polyphosphate compound is selected from the group consisting of 2-O-butyryl-1-O-octyl-myo-inositol 3,4,5,6-tetrakisphosphate octakis (propionoxymethyl) ester (INO-E2), 2-O-butyryl-3-O-octyl-myo-inositol 1,4,5,6-tetrakisphosphate octakis (propionoxymethyl) ester (INO-E3), 2-O-butyryl-1-O-(3-phenylpropyl)-myo-inositol 3,4,5,6-tetrakisphosphate octakis (propionoxymethyl) ester, 2,4,6-tri-O-butyryl-myo-inositol 1,3,5-trisphosphate hexakis (propionoxymethyl) ester, 1,2,5-tri-O-butyryl-myo-inositol 3,4,6-trisphosphate hexakis (propionoxymethyl) ester (ent-TMX/PM), 2,3,5-tri-O-butyryl-myo-inositol 1,4,6-trisphosphate hexakis (propionoxymethyl) ester (T/PM), 2,3-camphanylidene-myo-inositol 1,4,5,6-tetrakisphosphate octakis (propionoxymethyl) ester, 1,2-camphanylidene-myo-inositol 3,4,5,6-tetrakisphosphate octakis (propionoxymethyl) ester, inositol 3,4,5,6-tetrakisphosphate propionoxymethyl ester, inositol 1,4,5,6-tetrakisphosphate propionoxymethyl ester, D,L-2-O-butyryl-phosphatidylinositol 3,4,-trisphosphate heptakis(acetoxy)methyl ester (BtPIP₃/AM), 3,6-di-O-butyryl-myo inositol 1,2,4,5 -tetrakisphosphate octakis (propionoxymethyl) ester, and 1,4-di-O-butyryl-myo inositol 2,3,5,6-tetrakisphosphate octakis (propionoxymethyl) ester

5. The method of claim 4, wherein the sodium uptake inhibiting inositol polyphosphate compound is 1-octyl-2-O-butyryl-inositol 3,4,5,6-tetrakisphosphate propionoxymethyl ester (INO E2).

6. The method of claim 2, wherein the sodium uptake modulating inositol polyphosphate compound is a sodium uptake enhancing inositol polyphosphate compound.

7. The method of claim 6, wherein the sodium uptake enhancing inositol polyphosphate compound is 1,4-di-O-butyryl-inositol 2,3,5,6-tetrakisphosphate propionoxymethyl ester.
